(19) United States
(12) Patent Application Publication
(10) Pub. No.: US 2024/0100045 A1
(43) Pub. Date: Mar. 28, 2024

(54) EMULSION COMPOSITION AND USES THEREOF IN THE PREVENTION AND/OR TREATMENT OF SKIN DAMAGE CAUSED BY RADIATION

(71) Applicant: TARIAN PHARMA, GRASSE (FR)

(72) Inventors: Gareth WINCKLE, JUAN LES PINS (FR); Philippe ANDRES, PEYMEINADE (FR); Janusz CZERNIELEWSKI, VALLAURIS (FR)

(73) Assignee: TARIAN PHARMA, GRASSE (FR)

(21) Appl. No.: 18/264,289

(22) PCT Filed: Feb. 18, 2022
(86) PCT No.: PCT/EP2022/054044

§ 371 Date: Aug. 4, 2023

(30) Foreign Application Priority Data
Feb. 19, 2021 (FR) 2101621

(51) Int. Cl. A61K 31/498; A61K 9/107; A61K 9/127; A61K 47/22; A61K 47/36; A61P 17/02; A61P 17/04

(52) U.S. Cl. CPC A61K 31/498; A61K 9/107; A61K 9/1274; A61K 47/22; A61K 47/36; A61P 17/02; A61P 17/04

(57) ABSTRACT

A water-based composition in a form suitable for topical administration contains a vasoconstrictor and is in the form of an emulsion having liquid crystals. The vasoconstrictor is chosen from the group consisting of brimonidine or salts thereof, in a solvent-based phase. The solvent-based phase includes polyethylene glycol in combination with propylene glycol, a hydrophilic film-forming agent chosen from a polyvinylpyrrolidone/vinyl acetate copolymer, and polyvinylpyrrolidone in a non-crosslinked, crosslinked or acetate form, taken alone or in combination, glycerine, an emulsifier chosen from (i) the combination of PEG-75 stearate and glyceryl monostearate, and (ii) the combination of polyoxyethylene-20 sorbitan monosteearate (polysorbate-60) and cetostearyl alcohol, an oleic acid or an oleyl alcohol, and an oil phase suitable for obtaining the emulsion having liquid crystals.

### TECHNICAL FIELD

The invention relates to the field of pharmaceutical compositions in a form suitable for topical administration. It relates more particularly to a pharmaceutical composition comprising a vasoconstrictor such as brimonidine or salts thereof, as well as such a composition for use as a medicine, more particularly in the prevention and/or treatment of radiation damage to skin.

Types of radiation that can damage the skin include ultraviolet (UV), including UVA and UVB which can cause sunburn, rays in the visible range, infra-red radiation (IR), ionising radiation such as X-rays and alpha, beta or gamma radiation, and radiation composed of protons.

### STATE OF THE ART

One of the first effects of radiation exposure on tissue is erythema, an inflammatory response that causes dilation of blood vessels and reddening of the skin. This reaction is visible approximately 6-8 hours after exposure to UV and disappears after 36-48 hours.

Dermal application of a highly selective alpha2-adrenergic receptor agonist to the face is known to reduce erythema by means of direct cutaneous vasoconstriction. The main characteristic of cutaneous vasoconstriction is pallor; by reducing the diameter of arterioles and small vessels in the dermis, it brings about an immediate reduction in blood flow, producing as reduction in skin colour in particular.

MIRVASO® gel (0.5% w/w brimonidine tartrate) is thus indicated for the symptomatic treatment of facial erythema associated with rosacea in adults.

Brimonidine is more particularly known for being a highly selective alpha2-adrenergic receptor agonist. Brimonidine is 1,000 times more selective for alpha2-adrenergic receptors than for alpha1-adrenergic receptors.

Brimonidine has been shown to be useful in the treatment of erythema caused by acne rosacea and has been proposed for other skin disorders. See, for example, patent application U.S. Ser. No. 10/853,585, patent application U.S. Ser. No. 10/626,037, and patent application U.S. Ser. No. 12/193,098.

Brimonidine (tartrate) has a chemical stability suitable for topical administration and a solubility profile that offers different formulation options.

For example, we know that patent document WO2015013709, which describes the reduction and/or inhibition of radiation-induced skin hyperplasia by using brimonidine tartrate, is a concentration of 0.01-5% by weight, preferably 0.1-2% by weight, by topical administration, e.g in the form of emulsion. For example, it discloses a composition comprising an alcoholic phase with an ethyl ester copolymer base on PVM/MA, ethanol and dimethicone, an aqueous phase based on PVP/PA copolymer and an oil phase comprising oleth-20, cocamide MEA and steareth-16. The presence of ethanol in such a formulation makes it unsuitable in particular for the treatment of radiation-induced dermatitis, as it would cause tingling and a burning sensation in patients with damaged skin.

We also know that patent document US20110224216 describes a method of treating induced erythema, for example, by physical procedures, such as laser radiation, UV radiation, radiofrequencies, radiotherapy, light-emitting diodes, by topical administration of a composition particularly in the form of a cream particularly comprising brimonidine tartrate, PEG-300 and PEG-6 stearate.

Furthermore, we know that document WO2012075319 describes an oxymetazoline-based composition for the treatment of rosacea and rosacea-related symptoms, which additionally contains butylhydroxytoluene, PEG, glycol, PEG-32 stearate, cetostearyl alcohol and oleyl alcohol.

A topical product designed for therapeutic purposes usually consists of an active ingredient and excipients. During formulation, the choice of excipients is essential to guarantee the efficacy of the drug by making the active ingredients soluble and optimising their skin penetration, for the stability of the galenical form and its texture, for local tolerance and for patient compliance. As well as the optimisation of each of these individual elements, a complex and complementary challenge is to identify the optimal balance of these key factors to deliver a product that meets the needs of patients, healthcare professionals and regulating bodies.

It is generally difficult to create a release and cutaneous reservoirs with ionised active agents including salts, because they tend to penetrate the stratum corneum (a barrier composed mainly of lipids) less readily. Furthermore, such active agents tend to be rapidly cleared from viable tissues due to their solubility in water.

Brimonidine is in fact a hydrophilic molecule and thus has difficulty penetrating through the lipid stratum corneum.

On the other hand, once the stratum corneum has been crossed, brimonidine enters a hydrophilic medium (the epidermis, especially the granular layer and the basal layer, and then the dermis), and is then eliminated, thus reducing effectiveness in terms of vasoconstriction.

The structure of the barrier formed by the skin thus poses a real challenge to obtaining a topical formulation designed for application to the skin, allowing the active vasoconstrictor agent to pass the outer lipid layer without being eliminated quickly in the lower hydrophilic layers in order to produce a rapid, consistent and prolonged vasoconstrictor effect for a period of 16 hours or even 24 hours.

Furthermore, the concentration of active vasoconstrictor agent used must not be too high, as it would then pose a risk of significant and potentially harmful exposure at systemic level.

In addition, certain compounds used in compositions intended for topical application may cause side effects, which could limit their use and therefore their effectiveness. For example, certain active agents have the major disadvantage of causing irritation, which can lead to poor tolerance of the product. This in turn can result in non-compliance with treatment and dissatisfaction with the said treatment, on the part of the patient.

To this end, the formulation of MIRVASO® gel with its base of methyl parahydroxybenzoate, propylene glycol, carbomer, phenoxyethanol, glycerol, titanium dioxide, sodium hydroxide and purified water is, for example, not suitable for the prevention of lesions linked to radiation; such a formulation has non-optimal pharmacokinetics with limited activity from 6-12 hours after application. In addition, it contains particles of titanium dioxide which interfere with radiation when it is used for therapeutic purposes, for example in the prevention or treatment of radiation dermatitis.

There is currently a need to develop new compositions, making it possible to limit the effects linked to radiation and in particular the side-effects of treatment of cancer by radiotherapy.

Radio-dermatitis (or radiation-induced dermatitis) produces lesions that can be painful and worrisome for the patient, and can lead to temporary or permanent discontinuation of treatment.

There is however no consensus on the treatment of acute radio-dermatitis. A number of different solutions have been proposed, currently without sufficient levels of satisfaction to be adopted by all.

For acute grade 1 radiation dermatitis, emollients applied a few hours after the radiation session (such as DEXERYL® or TOPICREME®) moisturise the skin and bring a brief sense of well-being to the patient.

However, it is important to note that this option requires these products not to be applied before the session, to avoid a bolus effect (local increase in dose of radiation) and an increased risk of burning.

Some more specific products are offered for radio-dermatitis lesions, such as creams based on hyaluronic acid, TETA® cream or BIAFINE®. It is however recalled here that these treatments have not provided proof of effectiveness and that on the contrary, clinical studies have concluded that there is no effect.

Local topical corticosteroids (such as DIPROSONE®) should also be applied after the session. The theoretical principle of using these products is to reduce the inflammation caused by radiotherapy. Although topical corticosteroids do not provide real benefits for the development of radio-dermatitis, they are effective in cases of local allergic reaction (for example in cases of eczema linked to adhesives used for marking purposes).

In cases of acute radio-dermatitis, the continuation of radiotherapy treatment can be interrupted temporarily if the radiotherapist deems it necessary or preferable, depending on the progress of the treatment and the treatment priorities.

The use of one topical vasoconstrictor, epinephrine, has been described for the prevention of radiation dermatitis in patients with breast cancer undergoing radiation therapy (James F. Cleary et al., Significant suppression of radiation dermatitis in breast cancer patients using a topically applied adrenergic vasoconstrictor, Radiation Oncology, 2017).

However, such a product, which is an extemporaneous alcohol-based preparation that evaporates, needs to be applied just before the radiotherapy treatment, up to 20 minutes before.

Moreover, its effect is limited, most notably because of its excessively short duration of action. Only 50% of patients, therefore, showed a significant benefit in the study in question.

In these cancer treatments, which patients already find difficult to tolerate, side effects can be limiting and can interfere with the optimal course of treatment. There is therefore a real need for new effective formulations that make it possible to produce a powerful and prolonged protective effect and thus substantially reduce the cutaneous side effects of radiotherapy treatment.

There is therefore a need to develop new formulations aimed at producing a powerful, prolonged, controlled reduction in blood flow in the skin over time, limited to the application site and overcoming the above-mentioned shortcomings in terms of tolerance, effectiveness and compliance for patients undergoing radiation, in particular for cancer patients being treated with radiotherapy.

### Technical Problem

Considering the foregoing, one problem that the present invention proposes to solve consists in developing an optimised topical formulation based on a well-established vasoconstrictor, such as brimonidine tartrate and aimed at improving the duration and potency of activity of the vasoconstrictor in preventing and significantly reducing the main cutaneous side effects caused by radiation, most notably in the treatment of cancer by radiotherapy.

### Advantages Obtained

The Applicant has developed a new topical composition which improves the duration and potency of vasoconstriction by making the vasoconstrictor bioavailable in the dermis and epidermis for longer than 12-14 hours, to protect the skin against damage caused by radiation and more particularly against the cutaneous side effects of radiotherapy treatment, while avoiding any interference with the radiotherapy rays likely to reduce their effectiveness on the tumour being treated or the radiation field.

Combinations that are complex and difficult to provide containing polar solvents of low molecular weight (below 150 g/mol and preferably below 100 g/mol) and polar solvents of higher molecular weight (above 150 g/mol, preferably between 350-650 g/mol) have been developed in order to create a reservoir of brimonidine tartrate on the surface of the skin and in the upper layers of the stratum corneum. However, the formation of reservoirs of hydrophilic compounds is much more complex than for lipophilic agents, as the polar compounds are not distributed in the stratum corneum as easily as lipophilic compounds. In addition, hydrophilic compounds distribute themselves more freely in viable tissues and are removed by circulation of blood into the underlying local vascular system. An optimal and complex compositional balance must therefore be identified to modulate variables such as thermodynamics, residual surface solubility, solubility in the stratum corneum, penetration and persistence in viable tissues (traction effects/solvent drag).

In addition to the parameters described above, elements important for creating acceptable dosage forms and finished products were also considered when developing the compositions according to the invention. The compositions according to the invention have been centred on optimal solvent systems which facilitate dermal administration and provide adequate physical, chemical and microbiological stability, in addition to appropriate local tolerance and cosmetic elegance.

The optimised topical composition thus proposed by the Applicant improves the duration of the vasoconstriction process (from a period of at least 14 hours to 24 hours) as well as its power, without however disrupting the passage of the radiation through the skin and thus avoiding reductions in its efficacy.

The composition according to the invention will allow the creation of a reservoir of active polar vasoconstrictor agent in the stratum corneum, a phenomenon usually obtained only with lipophilic molecules such as corticosteroids, the polar molecules generally being rapidly “washed out” by the bloodstream. This allows persistence in the skin and therefore a maximum and rapid effect on each re-application. This kinetic allows a maximum preventative efficiency and offers flexibility of use to patients and radiotherapists.

The optimised composition, which is particularly suitable for radiotherapy treatment, helps promote patient compliance and maximise the effectiveness of anti-cancer treatment.

Also, the new topical formulation thus developed by the Applicant is well tolerated because it irritates very little or not at all compared to the compositions of the prior art, with improved skin penetration and increased solubility of brimonidine tartrate.

Finally, the pharmaceutical compositions according to the invention developed are also economical, easy and quick to prepare.

### Technical Solution

The first aim of the solution to this problem is a composition in a form suitable for topical administration, with a water base, comprising a vasoconstrictor, the said composition being in the form of an emulsion, preferably water-in-oil or oil-in-water, more preferably oil-in-water, comprising liquid crystals and said vasoconstrictor being chosen from brimonidine or salts thereof, in a solvent-based phase comprising:
polyethylene glycol in combination with propylene glycol;
a hydrophyilic film-forming agent chosen from a Polyvinylpyrrolidone/Vinyl Acetate copolymer, polyvinylpyrrolidone (PVP) in a non-crosslinked, crosslinked or acetate form, taken alone or in combination, preferably a Polyvinylpyrrolidone/Vinyl Acetate copolymer as a hydrophilic film-forming agent;
glycerine;
an emulsifier chosen from the combination of PEG-75 stearate and glyceryl monostearate and the combination of polyoxyethylene-20 sorbitan monosteearate (polysorbate-60) and cetostearyl alcohol;
an oleic acid or an oleyl alcohol, preferably an oleyl alcohol; and
an oil phase suitable for obtaining an emulsion, preferably water-in-oil or oil-in-water, more preferably oil-in-water, comprising liquid crystals.

The second aim is a composition according to the invention for use as a medicine.

### BRIEF DESCRIPTION OF THE DRAWINGS

The invention and the advantages deriving thereof will be better understood by reading the following description and the non-limiting methods of implementation, in relation to the annexed figures in which:

FIG. 1 represents the stability measurements using the LUMiSizer® for compositions 19-0155.0045 (Brij/Arlamol-based W/O emulsion), 19-0155.0065P (Brij/Arlamol-based W/O emulsion), 19-0155.0090 (Gelot 64-based W/O emulsion) and 19-0155.0091 (Polawax-based W/O emulsion).

FIG. 2 represents the stability measurements using the LUMiSizer® for the 19-0155.0044, 19-0155.0070P, 19-0155.0076/F2, 19-0155.0083, 19-0155.0086, 19-0155.0088 and 19-0155.0089 compositions.

FIG. 3 represents the viscosity measurements obtained with different compositions using the reference composition 19-0155-0090P/F3 under the process conditions carried out in the context of Example 5 and more particularly of Table 14.

FIG. 4 represents the skin whitening scores obtained with different W/O emulsion compositions 19.0155-0083/F1 (Gelot-64 based), 19.0155-0087/F1 (Gelot-64 based) and 19.0155.0086A/F1 (Polawax based).

FIG. 5 represents the skin whitening scores obtained with different emulsion compositions 19-0155.0091/F1 (Polawax-based), 19-0155.0089/F1 (Brij-based) and 19-0155-0090/F1 (Gelot-based).

FIG. 6 shows the skin whitening scores obtained with different active (comprising brimonidine tartrate) or non-active (comprising only the vehicle) oil-in-water emulsions.

FIG. 7 represents the skin whitening scores obtained with different active (comprising brimonidine tartrate, 19-0155-0102/F5) or non-active (comprising only the vehicle, 19.0155-0102P/F2) oil-in-water emulsion compositions.

FIG. 8 represents the average erythema scores obtained with different active (comprising brimonidine tartrate) or non-active (comprising only of the vehicle) oil-in-water emulsion compositions.

FIG. 9 represents the skin whitening scores obtained with reference to vasoconstrictor products.

FIG. 10 represents the skin whitening scores obtained with a modified MIRVASO composition 1.5% w/w of brimonidine tartrate (19-0155-0098/F1), a Norepinephine solution and MIRVASO® (0.5% w/w of brimonidine tartrate).

### DESCRIPTION OF EMBODIMENTS

The invention relates to a composition in a form suitable for topical administration, with a water-base, comprising a vasoconstrictor.

The topical composition according to the invention is characterised in that it is the form of an emulsion, preferably water-in-oil or oil-in-water, more preferably oil-in-water, having liquid crystals.

The liquid crystals are infinite aggregates of molecules which considerably improve solubility and facilitate emulsification.

From a microstructural point of view, the combination of a hydrophilic non-ionic emulsifier with an aliphatic fatty alcohol leads to a specific organisation in the continuous phase of the emulsion by producing liquid crystals surrounded by lamellar phases. The prevalence of lamellar phases is related to the excess of fatty alcohol(s).

Liquid crystal formulations offer several advantages as vehicles for topical compositions, including:
repairing the skin barrier;
delivery of moisture to the skin;
excellent tolerability;
improved skin release of certain active agents.

The topical composition according to the invention is characterised in that it comprises a vasoconstrictor chosen from brimonidine or salts thereof, in a solvent phase comprising:
polyethylene glycol in combination with propylene glycol;
a hydrophilic film-forming agent selected from a Polyvinylpyrrolidone/Vinyl Acetate copolymer (KOLLIDON VA 64®), polyvinylpyrrolidone (PVP) in an non-crosslinked, crosslinked or acetate form, taken alone or in combination, preferably a Polyvinylpyrrolidone/Vinyl Acetate copolymer as a hydrophilic film-forming agent;
glycerine;
an emulsifier chosen from the combination of PEG-75 stearate and glyceryl monostearate and the combination of polyoxyethylene-20 sorbitan monostearate (polysorbate-60) and cetostearyl alcohol;
an oleic acid or an oleyl alcohol, preferably an oleyl alcohol;
and an oil phase suitable for obtaining an emulsion, preferably water-in-oil or oil-in-water, more preferably oil-in-water, comprising liquid crystals.

The term “salts or pharmaceutically acceptable salts” refers to those salts of a compound of interest that are safe and effective for topical use in mammals and that possess a desired biological activity. Pharmaceutically acceptable salts include salts of acid or base groups present in the specified compounds. Pharmaceutically acceptable acid addition salts include, but are not limited to the following: salts of hydrochloride, hydrobromide, hydriodide, nitrate, sulphate, bisulphate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulphonate, ethanesulphonate, benzenesulphonate, p-toluenesulphonate and pamoate (that is, 1,1′-methylene-bis-(2-hydroxy-3-naphthoate)). Certain compounds used in the present invention can form pharmaceutically acceptable salts with various amino acids. Suitable base salts include, but are not limited to, salts of aluminium, calcium, lithium, magnesium, potassium, sodium, zinc and diethanolamine. For a review of pharmaceutically acceptable salts, see Berge et al., 66 J. PHARM. SCI. 1-19 (1977). In the present context, the term “hydrate” refers to a compound of interest, or a pharmaceutically acceptable salt thereof, which further comprises a stoichiometric or non-stoichiometric amount of water bound thereto by non-covalent intermolecular forces.

Preferably, the brimonidine used in the compositions according to the invention is brimonidine tartrate, although the salt form presents a challenge from a stability point of view for a water-in-oil or oil-in-water emulsion formulation.

Indeed, the salts can interact with the non-ionic surfactant and polymers and reduce their aqueous solubility and thus harm the physical stability of the semi-solid formulation. Conversely, the saline form of the active agent generates a relatively high aqueous solubility and advantageously allows the design and evaluation of aqueous-based formulations which can offer improved performance in terms of sensory and local tolerance.

Concentrations between 0.15% and 3.00% of brimonidine or salts thereof, preferably brimonidine tartrate, by weight of the total weight of the composition are preferably used to achieve efficacy and an improved duration of effect up to 24 hours after application while preventing any risk of systemic exposure.

Preferably, the composition according to the invention comprises brimonidine or salts thereof, preferably brimonidine tartrate at a concentration between 0.50% and 2.50% by weight of the total weight of the composition, preferably between 0.75% and 1.50% w/w, more preferably between 1.00% and 1.50% w/w, even more preferably 1.00% or 1.50% w/w.

The concentration of brimonidine, preferably brimonidine tartrate, and the dose thus applied is advantageously adapted according to the place of application.

Indeed, the barrier formed by the skin is thicker, in particular in terms of the stratum corneum to be passed through, on the feet and hands than on the scalp, with the rest of the body and in particular the chest having an intermediate thickness. For the same dose, therefore, the concentration preferably used is advantageously lower on the scalp, for example in the region of 0.15-0.5% w/w, compared to that used on the rest of the body, for example the chest with a concentration of 0.75-1.5% w/w, or even on the feet and hands, for example 1.5-3% w/w.

Preferably, the oil phase of the water-in-oil or oil-in-water emulsion composed of liquid crystals according to the invention comprises cetyl alcohol and stearyl alcohol, taken alone or in combination, and/or a triglyceride ester of saturated caprylic and capric fatty acids of coconut/palm kernel and glycerol of vegetable origin (Miglyon 812N), and a Polypropylene Glycol (PPG)-11 stearyl ether (Arlamol PS11E-LQ-[RD]), taken alone or in combination.

Preferably, the oil phase comprises a combination of cetyl alcohol and stearyl alcohol.

The oil phase of the water-in-oil or oil-in-water emulsion composition comprising liquid crystals according to the invention preferably comprises cetyl alcohol, stearyl alcohol and oleyl alcohol, taken in combination, at a concentration of between 1% and 15% by weight of the total weight of the composition, preferably between 2.5% and 10% w/w.

Preferably, the oil phase comprises a combination of triglyceride ester of caprylic and capric fatty acids saturated with coconut/palm kernel and glycerol of vegetable origin and PPG-11 stearyl ether.

The oil phase of the water-in-oil or oil-in-water emulsion composition with liquid crystals according to the invention preferably comprises the triglyceride ester of caprylic and capric saturated fatty acids with coconut/palm kernel and glycerol of vegetable origin and PPG-11 stearyl ether taken in combination at a concentration of between 5% and 10% by weight of the total weight of the composition.

The topical composition according to the invention is characterised in that it comprises polyethylene glycol (PEG) in combination with propylene glycol (PG).

Although low molecular weight polyethylene glycols (PEGs) are commonly used in topical products, mainly because they are generally effective solvents for many types of active ingredients, they are not necessarily the most effective excipients in terms of topical administration.

This is mainly related to their high polarity and molecular weight which limit their absorption through the skin. These properties limit the potential for the solvent to carry (pull) the active agent into the skin. This generally occurs when a solvent dissolves under the skin and transports the dissolved solute into the skin.

In addition, the high solubilising capacity of PEGs can lead to suboptimal thermodynamics for topical administration, and in cases of use at high concentrations, product transformation, often associated with evaporation of volatile components such as water, cannot be used to enhance release in the skin. When viewed holistically, these properties can reduce delivery efficiency, although high concentrations are possible; most of the applied dose of topical agents remains on the surface of the skin or is lost to the surroundings by contact transfer.

Increased delivery efficiency, fraction of the applied dose, may limit the need for increased doses to achieve targeted levels of dermal delivery and the need to use high PEG concentrations.

It has also been shown that the penetration and permeation of PEGs depends on their molecular weight.

Nevertheless, in the water-in-oil or oil-in-water emulsion compositions, according to the invention, PEGs are essential to the topical formulation.

PEGs of small molecular weight up to PEG-600, such as PEG-200, PEG-300, PEG-400, or PEG-400 SR are preferably used, more preferably PEG-400 and even more preferably PEG-400 SR beneficially favouring the stability and tolerance of the water-in-oil or oil-in-water emulsion composition according to the invention by limiting the irritation potential, eliminating polar impurities and thus reducing the interaction between the excipient and the active agent (brimonidine tartrate) and the subsequent breakdown of the active agent.

Although PEG-400 or PEG-400 SR has a relatively low penetration into the stratum corneum due to its molecular weight and high polarity (low partition coefficient), it is the PEG that is preferably used in the water-in-oil or oil-in-water emulsion composition, according to the invention to reduce the rate of precipitation of the active agent on the skin surface and in the upper layers of the stratum corneum, for superficial solubility. This favours the prolonged administration of brimonidine tartrate into the viable layers of the skin and specifically into the vascular system of the dermal plexus where the target site of the brimonidine tartrate is located. PEG-400 or PEG-400 SR has adequate solubility to promote better retention of brimonidine tartrate in solution on the skin surface and in the upper layers of the stratum corneum.

Preferably, the composition according to the invention comprises of PEG at a concentration of between 1% and 20% by weight of the total weight of the composition, preferably between 5% and 15%, more preferably 10%.

Propylene glycol (PG, 1,2-propanediol) is a clear, colourless, hygroscopic liquid that is widely used as a solvent and preservative in a variety of parenteral and non-parenteral pharmaceutical formulations.

PG is known to be a better general solvent than glycerine and dissolves a wide variety of materials, including corticosteroids, phenols, barbiturates, phenols, barbiturates, vitamins (A and D), most alkaloids and many local anaesthetics.

However, in the case of brimonidine tartrate, the PG has 50% of the solubility capacity of glycerine.

As an antibacterial agent, PG has a similar effect to ethanol; however, it is slightly less effective against moulds with a profile comparable to glycerine.

PG also exhibits volatility: although a fraction of the applied dose evaporates upon application to the skin or at least within 37 hours of application, a much larger portion penetrates the stratum corneum and penetrates the deep layers of the skin.

The relatively quick penetration of the PG through the stratum corneum and its volatility may deplete the residual vehicle of its solvent, increase the thermodynamic activity of the active agent in the vehicle and thus alter the driving force for diffusion. In addition, the penetration and permeation of PG can also disrupt the lipid barrier of the stratum corneum and thus reduce the diffusional resistance.

PG thus has favourable physiochemical properties in terms of skin penetration and permeation and is absorbed through the skin. Therefore, the solutes that are easily dissolved by PG (i.e. high affinity for the solvent/vehicle) may benefit from improved skin penetration via a solvent resistance mechanism or traction effects.

Even though data for various compounds are described in the literature as indicating dermal delivery of pharmaceutically relevant compounds can be enhanced by PG, it is not obvious for those skilled in the art to predict these characteristics in terms of stability, permeation efficiency of the active agent especially with brimonidine tartrate, and tolerability when it is used in a complex solvent system to obtain topical compositions according to the invention that are effective, stable and pharmaceutically acceptable.

A fortiori, PG is known to penetrate the skin more rapidly than most active agents and that therefore, precipitation of the active agent on the surface of the skin will limit its duration of action.

In addition, the concentrations of PG used in vivo are generally limited to about 20% w/w or less, to avoid local irritation reactions and problems of systemic toxicity.

The choice to use PG as a solvent and penetration enhancer in topical compositions according to the invention is not easily predictable, due to solubility and other vehicle-related variables.

Preferably, the composition according to the invention comprises PG at a concentration of between 5% and 40% by weight of the total composition weight, preferably between 10% and 30%, more preferably between 15% and 25%, and even more preferably 20%.

It is particularly beneficial in the context of water-in-oil or oil-in-water emulsion compositions according to the invention, to control PEG:PG ratio.

Excessively high concentrations of PEG in combination with PG, above 50% w/w, have negative effects in terms of the vasoconstriction intensity of brimonidine tartrate.

According to one preferred embodiment, PEG and PG are used in the ratio of 1:1 to 1:5, preferably 1:2.

Preferably, the composition according to the invention comprise spolyethylene glycol (PEG) at a concentration of 10% by weight of the total weight of the composition in combination with propylene glycol (PG) at a concentration of 20% by weight of the total weight of the composition.

The topical composition according to the invention is characterised in that it comprises a hydrophilic film-forming agent chosen from a Polyvinylpyrrolidone/Vinyl Acetate copolymer (PVP) in non-crosslinked, crosslinked or acetate form, taken alone or in combination.

Preferably, the composition according to the invention is comprised of the hydrophilic film-forming agent, taken alone or in combination, at a concentration of between 0.1% and 1.5% by weight of the total weight of the composition, preferably between 0.25% and 1.4%, more preferably between 0.5% and 1.3%, even more preferably between 0.75% and 1.25%, even more preferably at 1%.

Preferably, the topical composition according to the invention comprises a Polyvinylpyrrolidone/Vinyl Acetate copolymer (KOLLIDON VA 64®) as a hydrophilic film-forming agent.

Preferably, the composition according to the invention comprises the Polyvinylpyrrolidone/Vinyl Acetate copolymer (KOLLIDON VA 64®) at a concentration of between 0.1% and 1.5% by weight of the total weight of the composition, preferably between 0.25% and 1.4%, more preferably between 0.5% and 1.3%, even more preferably between 0.75% and 1.25%, even more preferably at 1%.

The topical composition according to the invention is characterised in that it further comprises glycerine.

Glycerine (glycerol) is a well-known humectant that can increase water retention in the stratum corneum and improve hydration.

Glycerine is also known and used to support the normal functioning of the skin barrier, promote skin elasticity and plasticity, improve skin smoothness and provide anti-irritant effects. Glycerine is in fact able to attract water into the stratum corneum from the epidermis and the atmosphere.

Due to its relatively high polarity, glycerine does not penetrate the skin to the same degree and depth as propylene glycol but can accumulate and form a reservoir in the hydrophilic regions of the stratum corneum and increase the water content.

The interaction of glycerine with the stratum corneum, its distribution in the skin, and its relatively high solubility for brimonidine tartrate (twice that of propylene glycol) provide advantages in terms of improved skin administration and prolonged skin penetration without causing stickiness on the skin surface.

Preferably, the composition according to the invention comprises glycerine at a concentration of between 1% and 20% by weight of the total weight of the composition, preferably between 2% and 15%, more preferably between 3% and 10%, even more preferably 4%.

In a particular advantageous way, the combination of PG and glycerine improves the distribution of the active agent, preferably brimonidine tartrate, in the stratum corneum.

The topical composition according to the invention is characterised in that it further comprises an emulsifier selected from the combination PEG-75 stearate and glyceryl monostearate and the combination of polyoxyethylene-20 sorbitan monostearate (polysorbate-60) and cetostearyl alcohol, preferably the combination of PEG-75 stearate and glyceryl monostearate (Gelot 64).

According to a preferred embodiment, the composition according to the invention, comprises the combination of PEG-75 stearate and glyceryl monostearate (Gelot 64) which contains fatty ingredients having a melting point between 46 and 66° C.

Considering the manufacturing process, the most important step after emulsification, which is done at a temperature of about 65±5° C., is the cooling stage. The decrease in temperature leads to the crystallisation of the fatty ingredients when they reach their transition temperature.

During the cooling stage, the fat emulsifiers/co-emulsifiers are organised around the oil droplets. The lipophilic co-emulsifier remains mainly in the oil droplets while the hydrophilic emulsifier and the amphiphilic fatty aliphatic alcohol remain at the interface between the oil droplets and the continuous aqueous phase of the emulsion.

Preferably, the composition according to the invention comprises the emulsifier e.g. GELOT 64® at a concentration of between 1% and 10% by weight of the total weight of the composition, preferably of between 2% and 7%, more preferably between 3% and 5%, even more of preferably at 3% w/w, or POLAWAX® at a concentration of between 3% and 15% by weight of the total weight of the composition, preferably of between 5% and 12%, more preferably of 10% w/w.

The topical composition according to the invention is characterised in that it further comprises an oleic acid or an oleyl alcohol, preferably an oleyl alcohol (KOLLICREAM OAC)).

Preferably, the composition according to the invention comprises the oleic acid or oleyl alcohol at a concentration of between 0.1% and 7% by weight of the total weight of the composition, preferably of between 1% and 5%, more preferably of 2.5%.

Preferably, the topical composition according to the invention further comprises xanthan gum as gelling agent.

Preferably, the composition according to the invention comprises xanthan gum at a concentration of between 0.1% and 1.5% by weight of the total weight of the composition, preferably between 0.2% and 1%, more preferably 0.2-0.5% for xanthan gum, respectively.

A soft mixed film is thus beneficially formed on the skin surface with xanthan gum and Polyvinylpyrrolidone/Vinyl Acetate copolymer (KOLLIDON VA 64®) in addition to the other non-volatile solvents, PG and PEG allowing to create a reservoir of brimonidine, preferably brimonidine tartrate, and thus slow down the precipitation of brimonidine and prolong its duration of action.

Preferably, the topical composition according to the invention further comprises a natural or synthetic antioxidant, or a free radical scavenger.

The antioxidant is preferably selected from butylated hydroxyanisole (BHA), DL-tocpherol, butylated hydroxytoluene (BHT), propaldehyde, ascorbate palmitate or glutathione, taken alone or in a mixture, preferably in a mixture, preferably BHA and/or DL-tocopherol.

The antioxidant is preferably used in the water-in-oil or oil-in-water emulsion compositions according to the invention at a concentration of between 0.01% and 4.0% by weight of the total weight of the composition, more preferably between 0.1% and 1.0%, even more preferably at 0.1%, for example BHA at 0.1% w/w and/or DL-tocopherol at 0.1% w/w.

Preferably, the topical composition according to the invention further comprises a paraben selected from methylparaben, propylparaben or isopropylparaben, taken alone or in combination.

Preferably, the composition according to the invention comprises paraben taken alone or in combination at a concentration of between 0.01% and 0.5% by weight of the total weight of the composition, preferably between 0.1% and 0.4%, more preferably at 0.3%.

Preferably, the composition according to the invention further comprises phenoxyethanol, preferably at a concentration of between 0.15% and 1.5%, more preferably between 0.4% and 1.25%, even more preferably between 0.5% and 1.1%, even more preferably 1% w/w; sodium benzoate, preferably at a concentration of between 0.05% and 0.5%, more preferably between 0.1% and 0.3%, even more preferably at 0.2% w/w, phenylethyl alcohol as an alternative preservative, preferably at a concentration of between 0.1% and 1%, more preferably between 0.25% and 0.75%, even more preferably 0.5% w/w; and/or EDTA as a chelating agent aiding the preservation and stability of the composition, preferably at a concentration of 0.2% w/w.

More preferably, the composition comprises phenoxyethanol, sodium benzoate, phenylethyl alcohol and EDTA taken in combination.

The incorporation of a hydrophilic solvent phase with solvents that have hygroscopic, humectant and skin conditioning properties, including PG and glycerine, improves the solubility of brimonidine tartrate in the stratum corneum and increases the water content therein.

The incorporation of antioxidants beneficially improves the stability of the active agent and the oil phase.

Also, the oil phases used have been selected to favour emulsion formation, physical stability, and the achievement of the desired microstructure, and to aid skin delivery while presenting appropriate sensory performance.

The water-in-oil or oil-in-water emulsion compositions, according to the invention, thus allows for improved and prolonged cutaneous delivery of brimonidine tartrate and meets the patients' needs with adequate and prolonged local vasoconstriction and superior epidermal and dermal protection from reactive oxygen species and inflammatory mediators.

Such compositions according to the invention are easy to apply and can be applied to potentially irritated skin.

They offer rapid drying with minimal residue on the skin.

The topical compositions according to the invention have a pH between 3.5.0 and 6.5, preferably between 4.0 and 5.5, more preferably 4.5.

The water-in-oil or oil-in-water emulsion compositions, according to the invention, have been designed to contain relatively high amounts of glycols, for example 20% PG and 10% PEG-400. Such relatively high concentrations of these components do not necessarily support stability and maintenance of the microstructure as they can disrupt the interface and solubilise the emulsifiers and co-emulsifiers. In addition, the addition of a relatively high concentration of an active salt, brimonidine tartrate, presents a possibility of physical destabilisation of the interface and the emulsion.

However, these characteristics have made it possible to obtain a water-in-oil or oil-in-water, preferably oil-in-water emulsion composition, with a high density of small oil droplets and liquid crystals thus improving the surface area of these structures and increasing the penetration of the active agent into the stratum corneum.

Another aim of the invention relates to a water-in-oil or oil-in-water, preferably oil-in-water, emulsion composition according to the invention for use as medicine.

Preferably, the water-in-oil or oil-in-water emulsion composition, according to the invention, is used for the prevention and/or treatment of damage caused by radiation, whether such radiation is photons, or protons and whether it is natural, therapeutic or accidental, including ultraviolet (UV) radiation, including UVA and UVB radiation which can cause sunburn, radiation in the visible range, infra-red (IR) radiation or ionising radiation such as X-rays and alpha, beta, gamma radiation or proton beams.

More preferably, the water-in-oil or oil-in-water emulsion composition, according to the invention, is used for the prevention and/or treatment of dermatitis resulting from radiation, notably within a radiotherapy treatment, e.g. by X-ray.

### EXAMPLES

This invention will now be shown with the following examples:

### Example 1: Measurement of Solubility Saturation in Pure Solvents

### Method

Saturated methods were prepared by adding an excess of drug substance to various solvents and storing the samples in sealed containers for 24 hours at room temperature with continuous stirring.

The majority of samples were stirred by a magnetic stirrer; however, in the case of viscous samples (e.g for pure glycerine), samples were stirred using a rotary mixer. The speed of the rotary mixer was adjusted to ensure proper mixing of the sample and this generally involved slower rotation speeds.

After the equilibration period, samples were centrifuged or filtered and the brimonidine tartrate was quantified using the Thermo Scientific Dionex U3000 UPLC-UV system. The chromatographic conditions are described below:• Column: Sunfire C18 150 mm×4.6 mm, 3.5 μm
Column temperature: 40° C.
Injection volume: 5111
Flow rate: 0.8m1/minute
UV Detection: 246 nm

### Results

The solubility saturation results for each solvent used independently are summarised in Table 1.

TABLE 1

| | | Max Solubility |
|---|---|---|
| Sample | Raw material | % (w/w) |
| E2 | TRANSCUTOL ® | 0.05 |
| E3 | ARLASOLVE ® DMI | 0.01 |
| E4 | GLUCAM ™ E10 | 0.72* |
| E5 | GLUCAM ™ E20 | 0.88* |
| E6 | Na Pyrrolidone Carboxylate (Ajidew L50) | 0.06 |
| E7 | Propylene Carbonate | 0.01** |
| E8 | PEG-400 | 0.10 |
| E9 | Hexylene Glycol | 0.01 |
| A1 | DMSO | ≥8.5*** |
| L1 | WATER (literature value^{‡}) | 3.4*** |
| | WATER (measured value) | 6.40 |
| L2 | Propylene glycol (literature value^{‡}) | ~0.1**** |
| | Propylene glycol (measured value) | 0.56 |
| | Glycerine (GLY, 20 ml scintillation bottle) | 1.61 |

*peak degradation observed after reinjection T + 72 hours
**peak degradation observed after reinjection T + 24 hours.
***visual solubility performed. Due to the high solubility observed, UPLC-UV analysis was not performed.
****Product Monograph: PrALPHAGAN ®, brimonidine tartrate, ophthalmic solution 0.2% w/v
^{‡}Conditions not specified

### Conclusion

According to the results obtained, DMSO appears to be an excellent solvent for brimonidine tartrate. Saturation was not reached even after the addition of 8.5% w/w brimonidine tartrate.

Water appears to be the second best solvent; the salt form of the active agent probably favours its solubility in water.

Then, in descending order, come glycerine (GLY), glucams, propylene glycol (PG) and PEG-400.

The observed solubility for other solvents is significantly lower and does not provide a satisfactory result for solubility of brimonidine tartrate.

Interestingly, well-known solvents such as Transcutol and DMI appear to be much less effective solvents than glycerine or propylene glycol for brimonidine tartrate.

### Example 2: Measurement of Solubility Saturation in Solvent Mixtures

### Method

Saturated solutions were prepared, incubated and measured as described in Example 1.

### Results

The solubility saturation results for different solvent mixtures are summarised in Table 2.

TABLE 2

| Mixing of raw materials | Max Solubility % (w/w) |
|---|---|
| PG/PEG-400 (ratio of raw materials: 20:10) | 0.45 |
| PG/PEG-400/GLY (20:10:5) | 0.66 |
| PG/PEG-400/PVP (20:10:1) | 0.45 |
| PG/PEG-400/GLY/(20:10:5:1) | 0.72 |
| PG/PEG-400/GLY/PVP/Water (20:10:5:1:40) | 3.3 |

### Conclusion

As expected, the non-aqueous solvent mixtures appear to have a lower solubility power than the mixtures containing water.

Nevertheless, according to the results thus obtained, it appears that the non-volatile mixture containing PG/PEG-400/GLY/PVP (20:10:5:1) was able to solubilise approximately 22% of the active agent compared to the aqueous mixture PG/PEG400/GLY/PVP/water (20:10:5:1:40). This indicates that the non-volatile polar components of an aqueous gel may have some ability to dissolve brimonidine tartrate on the surface of the skin and in the stratum corneum in the residual formulation even after the water has evaporated.

If concentrations of 1% or 1.5% brimonidine tartrate are used, the active agent may be at around 30% or 50% saturation respectively, in the aqueous solvent phase of the primary formulation prior to application. However, when the formulation is applied to the skin surface, the water will evaporate relatively quickly.

The results obtained therefore appear advantageous in terms of physical stability of the formulation but not necessarily in terms of conventional skin distribution, and the thermodynamic activity would be relatively low.

### Example 3: Measurement of Stability in Single Solvents and Preliminary Solvent Mixtures

The solvent combinations were prepared as described in Table 3. To facilitate evaluation, 0.1% brimonidine tartrate was added to each mixture. The samples were stored at ambient temperature, 40° C. and 50° C. for 1 month with sampling intervals of T=0 and T=1 month.

TABLE 3

| | Sample | | | | | | | | | |
|---|---|---|---|---|---|---|---|---|---|---|
| | M 1 | M 2 | M 3 | M 4 | M 5 | M 6 | M 7 | M 8 | M 9 | M 10 |
| Raw materials | (grams) | | | | | | | | | |
| DMSO | 25.0 | 25.0 | | | | 25.0 | 25.0 | | 25.0 | |
| GLUCAM E10 | 12.5 | | 12.5 | 12.5 | | 12.5 | 12.5 | | 12.0 | 12.5 |
| GLUCAM E20 | | 25.0 | | | 25.0 | 25.0 | 25.0 | | 25.0 | 25.0 |
| GLYCERINE | 12.5 | 12.5 | 12.5 | 12.5 | 12.5 | | | | 12.0 | |
| TRANSCUTOL | | | 50.0 | 25.0 | 50.0 | | 25.0 | 50.0 | 25.0 | |
| WATER | 100.0 | 87.5 | 75.0 | 100.0 | 62.5 | 87.5 | 62.5 | 100.0 | 50.0 | 112.5 |
| TOTAL | 150.0 | 150.0 | 150.0 | 150.0 | 150.0 | 150.0 | 150.0 | 150.0 | 150.0 | 150.0 |

The compatibility data generated in this study is summarised in Table 4.

TABLE 4

| | | T0 | | | T1 M | | | | |
|---|---|---|---|---|---|---|---|---|---|
| | | | Active | | | | Active | | Recovery |
| | | | agent | RSD | Storage | | Agent | RSD | relative to T0 |
| Reference | INCI | pH | (% w/w) | (%) | temperature | pH | (% w/w) | (%) | (%/T0) |
| M 1 | DMSO (10%) | 4.63 | 0.1035 | 0.1 | RT | 4.7 | 0.1060 | 1.0 | 102.4% |
| | GLUCAM E10 (5%) | | | | 40° C. | 4.87 | 0.1050 | 7.4 | 101.4% |
| | GLYCERINE (5%) | | | | 50° C. | | 0.0970 | 1.4 | 93.7% |
| M 2 | DMSO (10%) | 5.01 | 0.1044 | 0.9 | RT | 4.86 | 0.1010 | 1.4 | 96.7% |
| | GLUCAM E20 (10%) | | | | 40° C. | 4.92 | 0.0990 | 1.1 | 94.8% |
| | GLYCERINE (5%) | | | | 50° C. | | 0.0670 | 0.3 | 64.2% |
| M 3 | GLUCAM E10 (5%) | 5.09 | 0.1036 | 0.2 | RT | 5.03 | 0.1030 | 1.2 | 99.4% |
| | GLYCERINE (5%) | | | | 40° C. | 5.14 | 0.1010 | 1.1 | 97.5% |
| | TRANSCUTOL (20%) | | | | 50° C. | | 0.0970 | 1.3 | 93.6% |
| M 4 | GLUCAM E10 (5%) | 4.63 | 0.1013 | 0.3 | RT | 4.45 | 0.0990 | 2.1 | 97.7% |
| | GLYCERINE (5%) | | | | 40° C. | 4.76 | 0.0990 | 0.9 | 97.7% |
| | TRANSCUTOL (10%) | | | | 50° C. | | 0.0920 | 0.9 | 90.8% |
| M 5 | GLUCAM E20 (10%) | 5.29 | 0.0999 | 0.1 | RT | 5.18 | 0.1040 | 3.6 | 104.1% |
| | GLYCERINE (5%) | | | | 40° C. | 5.49 | 0.0970 | 0.7 | 97.1% |
| | TRANSCUTOL (20%) | | | | 50° C. | | 0.0770 | 0.6 | 77.1% |
| M 6 | DMSO (10%) | 5.42 | 0.1032 | 0.6 | RT | 5 | 0.1000 | 0.1 | 96.9% |
| | GLUCAM E10 (5%) | | | | 40° C. | 5.14 | 0.0920 | 0.5 | 89.1% |
| | GLUCAM E20 (10%) | | | | 50° C. | | 0.0450 | 0.2 | 43.6% |
| M 7 | DMSO (10%) | 5.86 | 0.1050 | 0.3 | RT | 5.87 | 0.1030 | 1.2 | 98.1% |
| | GLUCAM E10 (5%) | | | | 40° C. | 5.82 | 0.0990 | 0.9 | 94.3% |
| | GLUCAM E20 (10%) | | | | 50° C. | | 0.0900 | 2.9 | 85.7% |
| | TRANSCUTOL (10%) | | | | | | | | |
| M 8 | TRANSCUTOL (30%) | 4.46 | 0.1036 | 0.1 | RT | 4.28 | 0.1030 | 2.3 | 99.4% |
| | | | | | 40° C. | 4.7 | 0.1010 | 0.8 | 97.5% |
| | | | | | 50° C. | | 0.0980 | 1.6 | 94.6% |
| M 9 | DMSO (10%) | 5.93 | 0.0997 | 0.4 | RT | 5.93 | 0.0960 | 0.5 | 96.3% |
| | GLUCAM E10 (5%) | | | | | | | | |
| | GLUCAM E20 (10%) | | | | 40° C. | 5.89 | 0.0940 | 0.5 | 94.3% |
| | GLYCERINE (5%) | | | | 50° C. | | 0.0900 | 0.3 | 90.3% |
| | TRANSCUTOL (10%) | | | | | | | | |
| M 10 | GLUCAM E10 (5%) | 4.83 | 0.1034 | 0.7 | RT | 4.67 | 0.1000 | 0.0 | 96.7% |
| | GLUCAM E20 (10%) | | | | 40° C. | 4.47 | 0.0960 | 0.7 | 92.8% |
| | | | | | 50° C. | | 0.0430 | 2.0 | 41.6% |

According to the results obtained, the mean values are between 99.36% and 101.45% with a relative standard deviation of less than 1%.

The solvent mixtures without Glucam E20 showed the most favourable stability profile and

Solvent blends without Glucam E20 show the most favourable stability profile, while glycerine appears to improve stability. When Glucam E10 was included in the compositions at a concentration of 5%, the stability of brimonidine tartrate improved. Brimonidine tartrate measurement values tend to decrease as the Glucam concentration increases. The most stable mixture of solvents was obtained for M8 (30% Transcutol in water), then for M1 (10% DMSO, 5% Glucam E10 and 5% glycerine in water).

The data obtained indicate that using Glucam E10 and E20 increases the risk of brimonidine tartrate instability. On the other hand, transcutol, DMSO and glycerine show acceptable compatibility profiles.

### Example 4: Evaluation of Stability of Different Emulsion Compositions with the LUMiSizer® Technology

Different compositions were prepared and their short-term physical stability was evaluated.

Centrifugation and a more quantitative rapid screening tool, LUMiSizer®, were used to maximise efficiency and to facilitate the screening of different compositions.

The multi-sample LUMiSizer® is ideal for the characterisation and optimisation of dispersion stability, shelf life as well as particle-particle interactions, particle and flake compressibility, structural stability and elastic behaviour of sediments and gels.

Demixing phenomena are quantified in terms of clarification rate and instability index, sedimentation and particle flotation rate, residual turbidity, volume of separated phase (liquid or solid), sediment consolidation or dehydration.

The stability of the compositions mentioned in Tables 5-8 below was evaluated using a standardised LUMiSizer® protocol: 3-hour duration; temperature of 40° C. and centrifugation at 4000 rpm.

TABLE 5
19-0155.0045 (oil-in-water emulsion;
BRIJ/ARLAMOL-based; active agent)

| Ingredient | % w/w |
|---|---|
| Brimonidine tartrate | 1.5 |
| Purified water | 47.5 |
| Propylene Glycol | 20 |
| PEG-400 | 5 |
| GLUCAM E10 | 5 |
| GLYCERINE | 4 |
| KOLLOIDON VA-64 | 1 |
| BRIJ S2 (LOW HLB/LIPOPHILIC | 3 |
| SURFACTANT) | |
| BRIJ S721 (HIGH HLB/HYDROPHILIC | 2 |
| SURFACTANT) | |
| ARLAMOL PS11E: | 5.5 |
| LANETTE 18 | 2.5 |
| LANETTE 16 | 3.5 |
| DIMETHICONE FLUID 200 CST | 1 |
| KOLLICREAM OA | 2.5 |

TABLE 6
19-0155.0065P(oil-in-water emulsion; Brij/Arlamol base; vehicle)

| Ingredient | % w/w |
|---|---|
| Brimonidine tartrate | 0 |
| Purified water | 56.5 |
| Propylene Glycol | 20 |
| Glycerine | 4 |
| KOLLOIDON VA-64 | 1 |
| BRIJ S2 (LOW HLB/LIPOPHILIC SURFACTANT) | 3 |
| BRIJ S721 (HIGH HLB/HYDROPHILIC | 2 |
| SURFACTANT) | |
| ARLAMOL PS11E | 2.35 |
| KOLLIWAX SA | 2.5 |
| LANETTE 16 | 3.5 |
| DIMETHICONE FLUID 200 CST | 1 |
| KOLLICREAM OA | 2.5 |
| MARCOL 82 | 0.65 |

TABLE 7
19-0155.0090/F1(oil-in-water emulsion, Gelot 64 base)

| Ingredient | % w/w |
|---|---|
| Brimonidine tartrate | 1.5 |
| Purified water | 41 |
| Propylene Glycol | 20 |
| PEG-400 | 10 |
| Glycerine | 5 |
| KOLLOIDON VA-64 | 1 |
| XANTHAN GUM (XN) | 0.2 |
| GELOT 64 | 3 |
| ARLAMOL PS11E | 5 |
| MYGLIOL 812N | 2.5 |
| KOLLIWAX SA | 3 |
| KOLLIWAX CA | 4 |
| KOLLICREAM OA | 2.5 |

TABLE 8
19-0155.0091(oil-in-water emulsion, Polawax base)

| Ingredient | % w/w |
|---|---|
| Brimonidine tartrate | 1.5 |
| Purified water | 48.5 |
| Propylene Glycol | 20 |
| PEG-400 | 10 |
| GLYCERINE | 4 |
| KOLLOIDON VA-64 | 1 |
| XANTHAN GUM (XN) | 0.2 |
| POLAWAX NF: | 10 |
| ARLAMOL PS11E: | 2.35 |
| KOLLICREAM OA | 2.5 |

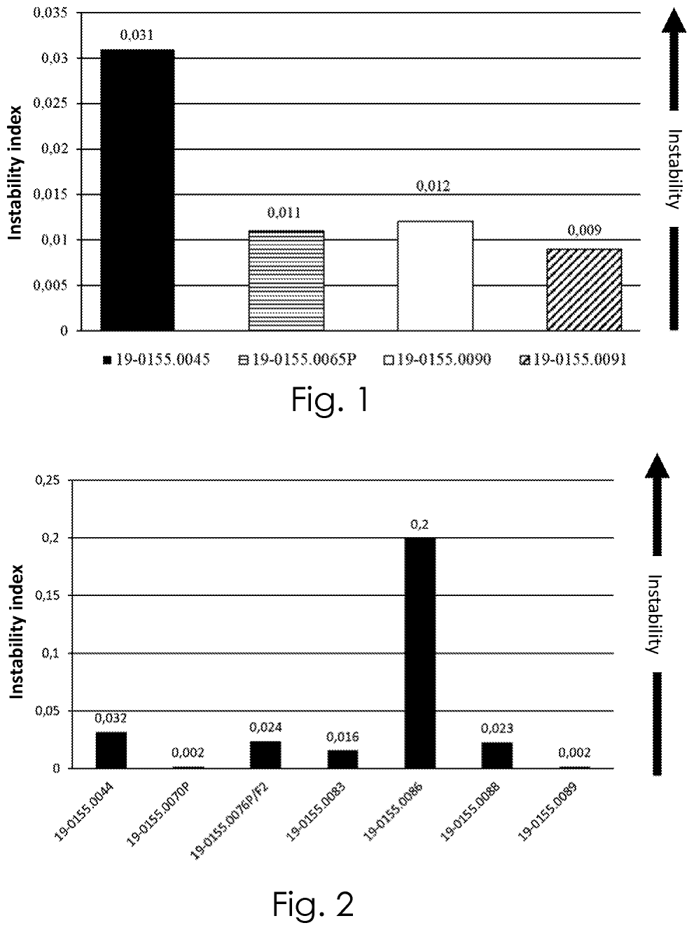

The results obtained with LUMiSizer® are shown in FIG. 1.

Such preliminary results show that the formulation 19-0155.0045 (oil-in-water emulsion with Brij/Arlamol base) is the least stable and less stable than the equivalent composition comprising only the vehicle (without active agent, brimonidine tartrate) (19-0155.0065P).

This data suggest that the active substance, brimonidine tartrate, has a destabilising effect, possibly due to its saline form, in this composition.

The other compositions comprising the active substance made from Gelot 64 (19-0155.0090) and Polawax (19-0155.0091), on the other hand, surprisingly show a physical stability similar to that of vehicle 19-0155.0065P.

Similarly complementary analyses on other compositions mentioned in Tables 9-11 were performed using LUMiSizer® technology.

TABLE 9
Brij/Arlamol E compositions examined using the LUMiSizer ® with associated preliminary
physical stability (19-0155.CR2.0044A/F1; 19-0155.0070P/F1, 19-0155.0076/F2 and 19-0155.0089/F1)

| | Formula-Batch No. | | | |
|---|---|---|---|---|
| | 19- | 19- | 19- | 19- |
| Composition | 0155.CR2.0044A/F1 | 0155.0070P/F1 | 0155.0076/F2 | 0155.0089/F1 |
| Concept | BRIJ | BRIJ | BRIJ | BRIJ |
| Batch size (g) | 420 (separate bulk | 200 | 200 | 200 |
| | 2 × 190 g) | | | |
| Justification of the | 0038A/F1 formula | 0065P formula | Process: | 65P Formula + |
| formula | without GLUCAM | with 0.3% | Deflocculating | 1.5% API + |
| | | Sepineo P600 | paddle (DP) | 10% PEG-400 + |
| | | | without Turaxx | Xanthane |
| | | | | formula |
| | | | | Process: DF |
| | | | | without Turaxx |
| Ingredient | % w/w | | | |
| Brimonidine Tartrate | 1.5 | 0 | 1.5 | 1.5 |
| Purified water | 52.5 | 56.2 | 45 | 44.8 |
| Propylene Glycol | 20 | 20 | 20 | 20 |
| Glycerine 4810 | 4 | 4 | 4 | 4 |
| Vegetale | | | | |
| PEG-400 | / | | 10 | 10 |
| KOLLIDON VA 64 | 1 | 1 | 1 | 1 |
| XANTHAN GUM | | | | 0.2 |
| BRIJ S2 | 3 | 3 | 3 | 3 |
| BRIJ S721 | 2 | 2 | 2 | 2 |
| ARLAMOL PS11E | 5.5 | 2.35 | 2.35 | 2.35 |
| LANETTE 18 | 2.5 | | | |
| KOLLIWAX SA | | 2.5 | 2.5 | 2.5 |
| LANETTE 16 | 3.5 | 3.5 | 3.5 | 3.5 |
| DIMETHICONE | 1 | 1 | 1 | 1 |
| FLUID 200 CST | | | | |
| KOLLICREAM OA | 2.5 | 2.5 | 2.5 | 2.5 |
| MARCOL 82 | | 0.65 | 0.65 | 0.65 |
| IP PARABENS | 1 | 1 | 1 | 1 |
| SEPINEO P600 | | 0.3 | | |
| NaOH Solution 10% | | QSPH 4.5 | | |
| Total | 100 | 100 | 100 | 100 |
| Macroscopic aspect T0 | Light yellow thick | Thick white | Light yellow | Light yellow |
| | cream | cream | thick cream | thick cream |
| T1M RT/40° C./50° C. | Idem T0/Idem T0/ | | | |
| | Phase separation | | | |
| T2M 4° C./RT/40° C. | | | Less yellow/ | |
| | | | inhomogeneous | |
| | | | light yellow | |
| | | | thick cream on | |
| | | | the walls/Idem | |
| | | | AT | |
| Microscopic aspect T0 | Fine emulsion | Fine emulsion, | N/A | Fine emulsion |
| | (droplets <3 μm), | (droplets up to | | (droplets up to |
| | some small LCs | 21 μm), many | | 14 μm), |
| | | LCs | | presence of |
| | | | | LCs |
| T1M AT | Idem T0 | N/A | N/A | N/A |
| T2M 4° C./RT/40° C. | N/A | N/A | T2M Idem T = 0/ | N/A |
| | | | Inhomogeneous | |
| | | | birefringence, | |
| | | | i.e LCs of the | |
| | | | esome/many | |
| | | | small LCs | |
| pH T0 | 5.32 | 4.81 | 4.2 | 4.2 |
| T1M RT/40° C. | 5.37/5.32 | N/A | N/A | N/A |
| Viscosity (RV mob 6, | 27300 cp/54.6% | N/A | N/A | N/A |
| speed 20-5 min) T0 | | | | |
| T7J RT | 23200 cp/46.4% | N/A | N/A | N/A |
| T2M RT | 24250 cp/48.5% | N/A | N/A | N/A |
| Comments | Emulsion: RT ok | No follow-up | Stability: RT, | Stability: RT, |
| | 40° C. | on stability but | macro: Light | macro: |
| | No increase in the | observations | yellow thick | heterogeneous |
| | viscosity | made: RT, | cream. x20: V. | cream Micro |
| | | macro: Thick | little LCs. | x20: Little LCs. |
| | | white cream. | 40° C.: macro: | 40° C.: ok |
| | | Micro x20: | possible | macro, micro: |
| | | LC <15μ No | exudate. micro: | many LCs 20μ |
| | | change at 40° C. | LCs + greater | |
| | | | than 76F1 | |

RT—Room Temperature
LC—liquid crystals
N/A—Not applicable

TABLE 10
Gelot 64 compositions examined using the LUMiSizer ® with associated
preliminary physical stability (19.0155.0083/F1 and 19.0155-0087/F1)

| | Formula-Batch No. | |
|---|---|---|
| Composition | 19.0155-0083/F1 | 19.0155-0087/F1 |
| Concept | GELOT (HA) | GELOT (HA) |
| Batch size (g) | 200 | 200 |
| Justification of the formula | Formula 0082 (Gelot 3%)+: | Formula 0085 (Gelot 3%) + |
| | deflocculating paddle (DP) | KOLLICREAM + |
| | for 5 min during | KOLLIDON; deflocculating |
| | emulsification. | paddle during the whole |
| | | process. |
| Ingredient | % w/w | |
| Brimonidine Tartrate | 1.5 | 1.5 |
| Purified water | 42.2 | 41.2 |
| Propylene Glycol | 20 | 20 |
| GLYCERINE 4810 | 5 | 5 |
| VEGETALE | | |
| PEG-400 | 10 | 10 |
| METHYLPARABEN | 0.2 | 0.2 |
| PHENOXYETHANOL | 1 | 1 |
| KOLLIDON VA 64 | | 1 |
| GELOT 64 | 3 | 3 |
| KOLLIWAX SA | 3 | 3 |
| MIGLYOL 812N | 5 | 2.5 |
| ARLAMOL PS11E-LQ- | 5 | 5 |
| (RB) | | |
| PROPYLPARABEN | 0.1 | 0.1 |
| KOLLICREAM OA | | 2.5 |
| LANETTE 16 | 4 | 4 |
| Total | 100 | 100 |
| Macroscopic aspect T0 | White to slightly yellow | White to slightly yellow |
| | shiny cream | shiny cream |
| T2M RT/40° C. | T2M Idem T0/Idem at RT, | T2M Idem T0/Idem at RT, |
| | slightly more yellow | slightly more yellow |
| Microscopic aspect T0 | Large number of LCs | Large number of LCs |
| T2M RT/40° C. | N/A | Fine emulsion (droplets 8- |
| | | 27 μm)/Idem RT, a lot of LCs |
| pH T0 | 4.2 | 4.1 |
| T2M RT/40° C. | 3.99/3.87 | 4.07/3.95 |
| Comments | Mixing by paddle with | Mixing by deflocculating |
| | holes - degassing required. | paddle, no degassing required. |
| | Stability: RT, macro: soft, | Stability: RT, macro: soft, |
| | shiny, slightly yellow cream. | shiny, slightly yellow cream. |
| | Micro x20: LCs ~15 μm | Micro x20: LCs <15 μm |

LC—liquid crystals

TABLE 11
Polawax NF composition examined using the LUMiSizer ® with
associated preliminary physical stability (19.0155-0086/F1)

| | Formula-Batch No. |
|---|---|
| Composition | 19-0155.0086/F1 |
| Concept | POLAWAX |
| Batch size (g) | 200 |
| Justification of the formula | 10% Polawax deflocculation paddle process |
| Ingredient | % w/w |
| Brimonidine Tartrate | 1.5 |
| Purified water | 46 |
| Propylene Glycol | 20 |
| Glycerine 4810 Vegetale | 4 |
| PEG-400 | 10 |
| KOLLIDON VA 64 | 1 |
| XANTHAN GUM | 0 |
| POLAWAX NF | 10 |
| ARLAMOL PS11E-LQ-(RB) | 2.35 |
| DIMETHICONE FLUID 200 | 1 |
| CST XIAMETER PMX-200 | |
| KOLLICREAM OA | 2.5 |
| MARCOL 82 | 0.65 |
| IP PARABENS | 1 |
| Total | 100 |
| Macroscopic aspect T0 | Slightly yellow, creamy fluid |
| T1M 4° C./RT/40° C. | N/A |
| T2M 4° C./RT/40° C. | No change T = 0/slightly mottling, small exudate |
| | on surface/Idem RT + colour change |
| T3M 4° C./RT/40° C. | T3M Idem T2M/Idem T2M/Idem T2M |
| Microscopic aspect T0 | N/A |
| T2M 4° C./RT/40° C. | Fewer LCs of RT T2M/LCs present with a certain |
| | number of less spherical examples/Normal light: |
| | thinner base vs RT; Polarised light: Rounder LCs |
| T3M 4° C./RT/40° C. | High number of LCs but some deformations/ |
| | Idem T3M at 4° C./Idem T3M at 4° C. |
| pH T0 | 4.14 |
| T1M 4° C./RT/40° C. | N/A |
| T2M 4° C./RT/40° C. | 4.03/4.20/4.10 |
| T3M 4° C./RT/40° C. | 3.95/4.18/3.94 |
| Comments | Stability: RT, macro: slightly yellow thick cream, |
| | matt surface, some mottled areas. Micro x20: |
| | some LCs below 0084/F1 (5-8 μm). /40° C.: |
| | exudate observed |

LC—liquid crystals

The additional results obtained are shown in FIG. 2.

Conventional visual observations and results obtained with the Lumisizer® indicate that the Polawax-based composition appears to be the least stable with phase separation observed after 3 months at 40° C.

Beneficial observations could be made from the BRIJ/ARLAMOL-based compositions:
The vehicle (19-0155.0070P/F1) is more stable than the same composition comprising the active agent brimonidine tartrate (19-0155.CR2.0044A/F1);
The addition of 10% w/w PEG-400 increases the stability of all tested compositions comprising active agent (19-0155.0076/F2 and 19-0155.0089/F1 vs 19-0155.CR2.0044A/F1);
The addition of xanthan gum also increases stability, with composition 19-0155.0089/F1 being slightly more stable than composition 19-0155.0076/F2 (showing an exudate after 3 months storage at 40° C. and a slightly higher instability index).

The two Gelot 64-based emulsions (19-0155-0083/F1 and 19-0155-0087/F1) show good stability even without the beneficial inclusion of xanthan gum as a stabiliser and give the best results.

The addition of polymers was also tested in similar additional analyses with the compositions detailed in Table 12 below using the Gelot 64-based composition (19-0155.0087/F1) as a reference.

TABLE 12
Compositions tested for the evaluation of polymer stabilisation using LUMiSizer ®

| | Formula-Batch No. | | |
|---|---|---|---|
| Composition | 19.0155-0087/F1 | 19.0155-0103/F1 | 19.0155-0104/F1 |
| Concept | GELOT | GELOT | GELOT |
| Batch size (g) | 200 | 200 | 200 |
| Justification of the formula | Formula 0085 | Equivalent to the | Formula 0087 + 1.5% |
| | (Gelot 3%) + | formula of vehicle 102P + | Brimonidine; 1% BHA + |
| | KOLLICREAM + | Brimonidine. Change | 0.3% Sepineo P600 |
| | (KOLLIDON, | of preservation system; | |
| | PVP); | Xanthan gum 0.2% | |
| | deflocculating | | |
| | paddle during the | | |
| | whole process | | |
| Ingredient | % w/w | | |
| Brimonidine Tartrate | 1.5 | 1.5 | 1.5 |
| Purified water | 41.2 | 40.3 | 40.2 |
| Propylene Glycol | 20 | 20 | 20 |
| Glycerine 4810 Vegetale | 5 | 5 | 5 |
| PEG-400 | 10 | 10 | 10 |
| METHYLPARABEN | 0.2 | | |
| PHENOXYETHANOL | 1 | | |
| KOLLIDON VA 64 | 1 | 1 | 1 |
| XANTHAN GUM | | 0.2 | |
| SEPINEO P600 | | | 0.3 |
| GELOT 64 | 3 | 3 | 3 |
| KOLLIWAX SA | 3 | 3 | 3 |
| LANETTE 16 | 4 | 4 | 4 |
| MIGLYOL 812N | 2.5 | 2.5 | 2.5 |
| ARLAMOL PS11E-LQ-(RB) | 5 | 5 | 5 |
| KOLLICREAM OA | 2.5 | 2.5 | 2.5 |
| BHA | 0 | 1 | 1 |
| IP PARABENS | | 1 | 1 |
| PROPYLPARABEN | 0.1 | | |
| Total | 100 | 100 | 100 |
| Macroscopic aspect T0 | White to slightly | White to slightly yellow | White to slightly yellow |
| | yellow shiny cream | shiny cream that does | shiny cream that does not |
| | | not run | run |
| T1M 4° C./RT/40° C. | N/A | T1M Idem T0/Idem | T1M Idem T0/Idem |
| | | T0/Slightly more yellow | T0/Idem T0 |
| T2M 4° C./RT/40° C. | T2M Idem | T2M Idem T0/Idem | T2M Idem T0/Idem |
| | T0/Idem RT, | T0/Idem T1M | T0/Slightly more yellow |
| | slightly more | | |
| | yellow | | |
| T3M 4° C./RT/40° C. | N/A | Idem T0/Homogeneous, | N/A |
| | | smooth, | |
| | | shiny/Homogeneous | |
| Microscopic aspect T0 | Important number | Fine emulsion with a | Fine emulsion with a large |
| | of LCs | large number of LCs. | number of LCs. Same as |
| | | Same as the | the placebo/vehicle. |
| | | placebo/vehicle. | |
| T1M 4° C./RT/40° C. | | Idem T0/Idem T0/Idem | LCs 8-28 μm/Same at |
| | | T0 | 4° C./High LC density |
| T2M 4° C./RT/40° C. | Fine emulsion | T2M Idem T0/Slightly | Heterogeneity of LC |
| | (droplets 8-27 | lower density of small | size/LC deformation/ame |
| | μm)/Idem RT, a | LCs compared to T0, | as T1M |
| | lot of LCs | some larger LCs/Idem | |
| | | than T2M 4° C. | |
| T3M 4° C./RT/40° C. | | Idem/Idem T0/Idem | |
| | | T2M | |
| pH T0 | 4.1 | 4.54 | 4.27 |
| T1M 4° C./RT/40° C. | N/A | T1M 4.01/4.03/3.93 | T1M 3.96/4.12/3.86 |
| T2M 4° C./RT/40° C. | N/A/4.07/3.95 | T2M 3.95/4.00/3.88 | T2M 3.99/3.91/3.87 |
| T3M 4° C./RT/40° C. | | T4M 4.07/4.08/4.19 | |
| Comments | Mixing by | | |
| | deflocculating | | |
| | paddle, no | | |
| | degassing required. | | |
| | (Stability: RT, | | |
| | macro: soft, shiny, | | |
| | slightly yellow | | |
| | cream. Micro x20: | | |
| | LCs <15 μm | | |

LC—liquid crystals

The additional results obtained show that the addition of xanthan gum (0.2% w/w) drastically increases the stability of emulsion 19-0155.0103/F1 compared to reference formulation 19-0155.0087/F1.

On the other hand, the incorporation of an ingredient such as SEPINEO P600® destabilises emulsion 19-0155.0104/F1 such that the stability is significantly lower than that of reference formulation 19-0155.0087/F1.

### Example 5: Evaluation of the Impact of Varying Different Process Steps in the Manufacture of an Oil-In-Water Emulsion Composition

The vehicle formulated in Table 13 was used to assess the impact of varying different manufacturing process steps on the composition in terms of the appearance and stability. The initial characteristics of such a vehicle in terms of appearance, pH, and viscosity are also described below.

TABLE 13
Composition of vehicle prototypes 19-0155-0090P/F3 and
physical parameters for composition 19-0155-0090P/F3

| | Formulation ID |
|---|---|
| | 19-0155-0090P/F3 |
| Ingredient | % w/w |
| Methylparaben | 0.2 |
| Phenoxyethanol | 1 |
| Propylene Glycol | 20 |
| Glycerine 4810 Vegetale | 5 |
| PEG-400 | 10 |
| Purified water | 42.5 |
| Brimonidine Tartrate | 0 |
| KOLLIDON VA 64 | 1 |
| XANTHAN GUM | 0.2 |
| GELOT 64 | 3 |
| KOLLIWAX SA | 3 |
| MIGLYOL 812N | 2.5 |
| ARLAMOL PS11E-LQ-(RB) | 5 |
| PROPYLPARABEN | 0.1 |
| KOLLICREAM OA | 2.5 |
| KOLLIWAX CA | 4 |
| Total | 100 |
| Parameter | Result |
| White to off-white appearance, | shiny cream and thick |
| pH | 4.92 |
| Viscosity (RV29/30 rpm) | 13770 cP/41.3% |

The results obtained for the different process variations are described in Table 14.

TABLE 14
Summary of process variables assessed using the base composition 19-0155-0090P

| | Formulation #: 19-0155-00XXP/FY. | | | | | | |
|---|---|---|---|---|---|---|---|
| Details | 90P/F3 | 90P/F4 | 90P/F5 | 90P/F6 | 136P/F1 | 136P/F2 | 90P/F7 |
| Deflocculating | X | X | | X | X | | |
| process | | | | | | | |
| Croda process | | | X | | | X | X |
| Long cooling time | X | X | | | | | |
| Short cooling time | | | X | X | X | X | X |
| Phenoxy | | X | X | X | X | X | X |
| introduced after | | | | | | | |
| emulsion | | | | | | | |
| Removal of | | | | | X | X | |
| glycols | | | | | | | |
| Glycols | | | | | | | X |
| introduced after | | | | | | | |
| emulsion | | | | | | | |
| “Active” phase | | | | | | | |
| introduced at RT | | | | | | | |
| after emulsion | | | | | | | |
| Silicone | | | | | | | |
| introduction | | | | | | | |
| “Active” phase | | | | | | | |
| introduced at | | | | | | | |
| 45° C. after | | | | | | | |
| emulsion | | | | | | | |
| pH change | X | X | X | X | X | X | |
| pH | 4.92 | 4.88 | 5.07 | 4.54 | 5.01 | 5.19 | 5.78 |
| Viscosity | 13,770 | 10,770 | 20,400 | | 8,167 | 11,200 | 13,430 |
| (Mob29/30 rpm) | | | | | | | |
| Comments | Source formula: | Lumisizer: | Very, very | Comparison/ | Glycol removal | The Croda | Comparison/F5: |
| | homogeneous | different | fine base, | F4 => valid | significantly | process | globule |
| | globule | profile | heterogeneous | introduction of | increases | refines the | magnification |
| | dispersion of | | LCs | phenoxy after | globule size | globules. | |
| | size <10 μm. | | | emulsion and | | | |
| | | | | short cooling time | | | |

| | Formulation #: 19-0155-00XXP/FY. | | | | | |
|---|---|---|---|---|---|---|
| Details | 90P/F8 | 90P/F9 | 90P/F10 | 90P/F11 | 90P/F12 | 90P/F13 |
| Deflocculating | | X | X | | | x Rotor-stator |
| process | | | | | | |
| Croda process | X | | | X | X Turrax at | |
| | | | | | the end | |
| Long cooling time | | | | | | |
| Short cooling time | X | X | X | X | X | X |
| Phenoxy | X | X | X | X | X | X |
| introduced after | | | | | | |
| emulsion | | | | | | |
| Removal of | | | | | | |
| glycols | | | | | | |
| Glycols | | | | | | |
| introduced after | | | | | | |
| emulsion | | | | | | |
| “Active” phase | X | X | | | | |
| introduced at RT | | | | | | |
| after emulsion | | | | | | |
| Silicone | | | X | | | |
| introduction | | | | | | |
| “Active” phase | | | | X | X | |
| introduced at | | | | | | |
| 45° C. after | | | | | | |
| emulsion | | | | | | |
| pH change | | | | | | |
| pH | 5.69 | 5.57 | 5.54 | 5.6 | 5.6 | 5.52 |
| Viscosity | 8100 | 8467 | 10,700 | 6267 | 6667 | 8367 |
| (Mob29/30 rpm) | | | | | | |
| Comments | Comparison/F5: | Poorly formed, | Soaping | Do not heat the | Thickens | Refines |
| | Lumisizer with | heterogeneous | eliminated. | active phase => fatty | the formula | Lumisizer OK a |
| | different profile, | globules. | | alcohol clusters. | but | lot |
| | more homogeneous | | | | improves | |
| | globules and visco | | | | little. | |
| | impact | | | | | |

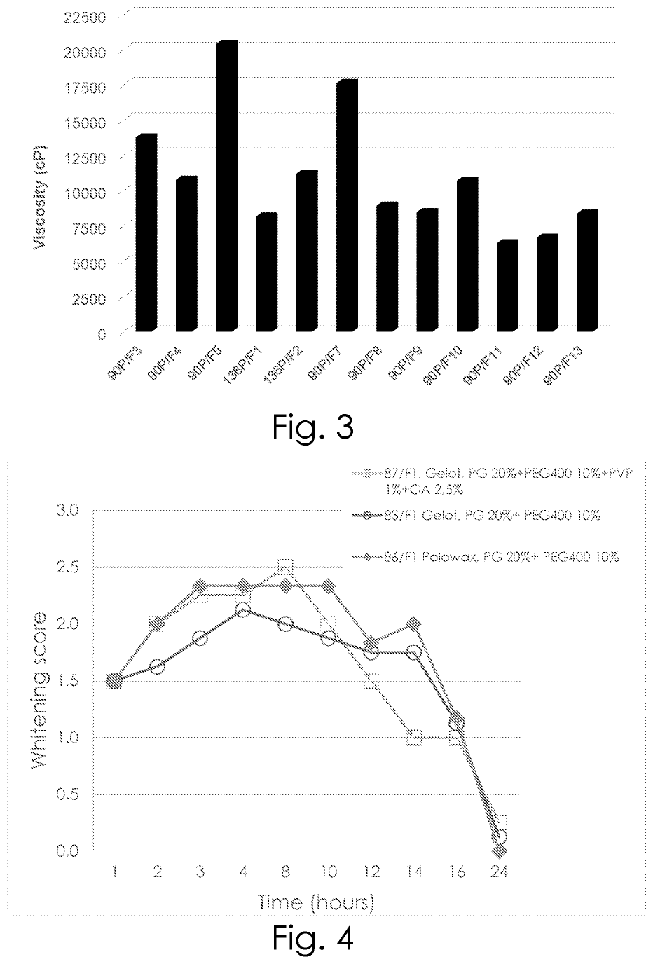

The results in terms of viscosity are shown in FIG. 3.

The “deflocculator” and “Croda” processes, in particular, described below have been tested comparatively.

The “deflocculator” process uses a deflocculating or dispersing blade, whereas a paddle stirrer is used in the “Croda” process throughout the manufacturing process. “Deflocculator” process

Phase A:

Equipment used: beaker: 600m1; type of stirring: deflocculating or dispersing blade

Step 1: Weigh and add water, PG and PEG-400 to the beaker.

Heat to 70° C. and mix with the deflocculating or dispersing blade until homogenisation.

Step 2: Weigh and add KOLLIDON to the mixture obtained in step 1. Mix with the deflocculating or dispersing blade until homogenisation.

Step 3: Add xanthan gum previously dispersed in the glycerine to the mixture obtained in step 2.

Step 4: When the content of the mixture obtained in Step 3 is homogeneous, weigh and add the methylparaben.

Step 5: No more than 20 minutes before emulsification, phenoxyethanol is introduced into the mixture obtained in step 4.

Phase B:

Equipment used: another beaker; type of stirring: magnetic bar

Step 6: Weigh and add all excipients constituting the fatty phase. Heat to 70° C. and mix until homogenised.

Emulsification Phase:

Type of stirring: deflocculating or dispersing blade

Step 7: At 70° C., pour the phase B mixture obtained in step 6 into the phase A mixture obtained in step 5 while mixing for 10 minutes at 500 rpm.

Cooling Step:

Type of stirring: deflocculating or dispersing blade

Step 8: Remove the mixture obtained in step 7 from the hot plate, allow to cool down to 35° C. while stirring at 200-300 rpm.

Step 9: Between 30° C. and 35° C., the formulation increases in viscosity and a spatula is used to remove the product from the walls of the beaker.

Step 10: Once the mixture obtained in step 9 is homogeneous, adjust to approximately pH 4.5-5 using a 10% citric acid solution.

Step 11: Once the pH has been adjusted, mix using the deflocculating/dispersing blade for 20 minutes at approximately 200 rpm.

Step 12: Once step 11 is done, complete with water (qs).

“Croda” process

Phase A:

Equipment used: beaker: 600m1; Type of stirring: paddle stirrer.

Step 1: Weigh and add water and Methylparaben to the beaker.

Heat to 70° C. and mix with the paddle stirrer until homogenisation.

Step 2: Weigh and add KOLLIDON to the mixture obtained in step 1. Mix with the paddle shaker until homogenisation.

Step 3: Add xanthan gum previously dispersed in the glycerine to the mixture obtained in step 2 at 65° C.

Phase B:

Equipment used: another beaker; type of stirring: magnetic bar

Step 4: Weigh and add all excipients constituting the fatty phase. Heat to 70° C. and mix until homogenised.

Emulsification phase:

Type of stirring: Ultra Turrax

Step 5: 70° C., pour the phase B mixture obtained in step 4 into the phase A mixture obtained in step 3 while mixing for 2 minutes at 11,500 rpm.

Cooling step:

Type of stirring: paddle stirrer (with holes)

Step 6: Remove the mixture obtained in step 5 from the hot plate, allow to cool down to 35° C. while stirring at 200-300 rpm.

Step 7: While cooling, introduce the PG and PEG-400 into the mixture obtained in step 6.

Step 8: At 35° C.-40° C., weigh and add phenoxyethanol to the mixture obtained in step 7.

Step 9: At around 30° C., the formulation increases in viscosity and a spatula is used to remove the product from the walls of the beaker.

Step 10: Once the mixture obtained in step 9 is homogeneous, adjust to approximately pH 4.5-5 using a 10% citric acid solution.

Step 11: Once the pH has been adjusted, mix using the paddle shaker for 30 minutes at approximately 200 rpm.

Step 12: Once step 11 is done, complete with water (qs).

The best results were obtained for the reference formula with the “deflocculator” process.

Appropriate viscosity and a high density of monodispersed liquid crystals/lamellar gel phases were obtained with this process for formulation 19-0155-0090P/F3.

The microstructure obtained under these conditions is the most effective for drug delivery because the droplet and liquid crystal/lamellar gel phases are small and of high density.

Such a microstructure maximises the specific surface area of the liquid crystal/lamellar gel phases to facilitate the transfer of the active agent into the skin and thus improve vasoconstriction in terms of vasoconstriction intensity and prolonged duration of vasoconstriction.

The droplet size is therefore influenced by the process used and the “Croda” process tends to reduce the droplet size to a greater extent than the “Deflocculator” process. This is probably due to the use of a high shear mixer during the emulsification process and the subsequent use of a paddle stirrer.

The cooling rate was also identified as an important variable and the ambient air cooling produced the most satisfactory results of the test carried out.

Several other observations could therefore be made with this test:

Phenoxyethanol should preferably be introduced after the emulsification step.

If the active phase (including the active agent) is introduced after emulsification, it should not be heated.

The microstructure was significantly influenced by the removal of glycols with a variation in droplet size and the liquid crystal/lamellar gel phase size with the “Deflocculator” and “Croda” processes.

The incorporation of 1% w/w dimethicone in the “deflocculator” process (formula 0090P/F10), appears to eliminate the soaping/whitening effect that was observed when rubbing/applying the formulation.

The microstructure does not appear to be significantly affected by this minor formulation change when manufactured using the deflocculator process.

### Example 6: Evaluation of the Effect on Skin Whitening of Different Compositions Using an In Vivo Vasoconstriction Model

Each of the compositions was triple tested using the in vivo vasoconstriction protocol as described below.

60 microlitres of each composition were applied once in a randomised blind fashion at 8 am to the upper chest, using a positive displacement pipette, on a 10 cm2 surface area defined with plastic O-rings.

After application, a 30-second massage period and a 10-minute post-application drying period of the product were then performed.

The whitening scores on a conventional scale of 0 to 3 (3=maximum) are measured 1, 2, 3, 4, 8, 10, 12, 14, 16 and 24 hours after application.

Two Gelot 64-based emulsions (Table 10 described above) and one Polawax NF-based emulsion (Table 11 described above) were evaluated.

The skin whitening results are shown in FIG. 4.

All emulsions tested generate a similar onset of action.

The Polawax NF emulsion (19-0155,0086A/F1) produced a slightly better overall profile than the Gelot 64-based compositions (19-0155-0083/F1 and 19-0155-0087/F1). It should be noted that composition 19-0155.0086A/F1 also contains oleyl alcohol (KOLLICREAM OA, 2,5%) and KOLLIDON VA-64, both of which improve and prolong the cutaneous administration of the locally applied active agent.

The Gelot-64 based composition (19-0155-0083/F1) does not contain any polymer or oleyl alcohol, whereas Gelot 64-based composition 19-0155-0087/F1 contains KOLLIDON VA-64 and oleyl alcohol. Both Gelot 64-based compositions showed superior physical stability compared to the Polawax-based formula.

Based on these results, it appears that the combination of xanthan gum and KOLLIDON VA 64 plus oleyl alcohol improves the skin administration of the active agent while providing the required physical stability.

Such oil-in-water emulsions have therefore demonstrated improved performance characteristics in terms of onset, intensity and duration of skin whitening.

### Example 7: Evaluation of the Effect on Skin Whitening of Different Compositions Using an In Vivo Vasoconstriction Model

Different formulations were thus tested and the results were divided into two groups: O/W emulsion [oil-in-water] (Gelot 64, Table 14) and O/W emulsion (Polawax, Table 15). Also, the formulations tested all include xanthan gum which has advantages in terms of stability.

TABLE 14
Composition of Gelot 64 cream 19.0155-0090/F1

| | Formula-Batch No. |
|---|---|
| Composition | 19.0155-0090/F1 |
| Concept | GELOT |
| Batch size (g) | 200 |
| Justification of the formula | Formula 0087 + Xanthan gum to |
| | increase stability. Deflocculating |
| | blade used throughout the process |
| Ingredient | % w/w |
| Brimonidine Tartrate | 1.5 |
| Purified water | 41 |
| Propylene Glycol | 20 |
| Glycerine 4810 Vegetale | 5 |
| PEG-400 | 10 |
| KOLLIDON VA 64 | 1 |
| XANTHAN GUM | 0.2 |
| METHYLPARABEN | 0.2 |
| PHENOXYETHANOL | 1 |
| GELOT 64 | 3 |
| KOLLIWAX SA | 3 |
| MIGLYOL 812N | 2.5 |
| ARLAMOL PS11E-LQ-(RB) | 5 |
| PROPYLPARABEN | 0.1 |
| KOLLICREAM OA | 2.5 |
| LANETTE 16/KOLLIWAX CA | 4 |
| Total | 100 |
| Macroscopic aspect T0 | White to off-white cream |
| Microscopic aspect T0 | Fine emulsion, droplets ~15 μm |
| Comments | Stability: RT, macro: slightly yellow |
| | soft cream. Micro x20: LCs 5 μm No |
| | change at 40° C. Slight increase |
| | in yellow colouration at 40° C. |
| | Micro: RT and 40° C. = |
| | identical. Reduction in average LC size. |

LC—liquid crystals

TABLE 15
Polawax composition 19-0155.0091/F1

| | Formula-Batch No. |
|---|---|
| Composition | 19-0155.0091/F1 |
| Concept | POLAWAX |
| Batch size (g) | 200 |
| Justification of the | 10% Polawax |
| formula | deflocculation paddle |
| | process + xanthan gum |
| Ingredient | % w/w |
| Brimonidine Tartrate | 1.5 |
| Purified water | 45.8 |
| Propylene Glycol | 20 |
| Glycerine 4810 | 4 |
| Vegetale | |
| PEG-400 | 10 |
| KOLLIDON VA 64 | 1 |
| XANTHAN GUM | 0.2 |
| POLAWAX NF | 10 |
| ARLAMOL PS11E- | 2.35 |
| LQ-(RB) | |
| XIAMETER PMX-200 | 1 |
| DIMETHICONE | |
| FLUID 200 CST | |
| KOLLICREAM OA | 2.5 |
| MARCOL 82 | 0.65 |
| IP PARABENS | 1 |
| Total | 100 |
| Macroscopic aspect T0 | Slightly yellow shiny cream |
| Microscopic aspect T0 | Fine emulsion, |
| | droplets ~15 μm |
| Comments | Stability: RT, macro: soft, |
| | slightly yellow shiny cream. |
| | RT Micro x20: LCs 5 μm. |
| | 40° C.: Idem T0 |
| | After 6 months (2015 July). |
| | Slight increase in yellow |
| | colouration at 40° C. Micro: |
| | RT and 40° C. = Idem. |
| | Reduction in average LC |
| | size. |

LC—liquid crystals

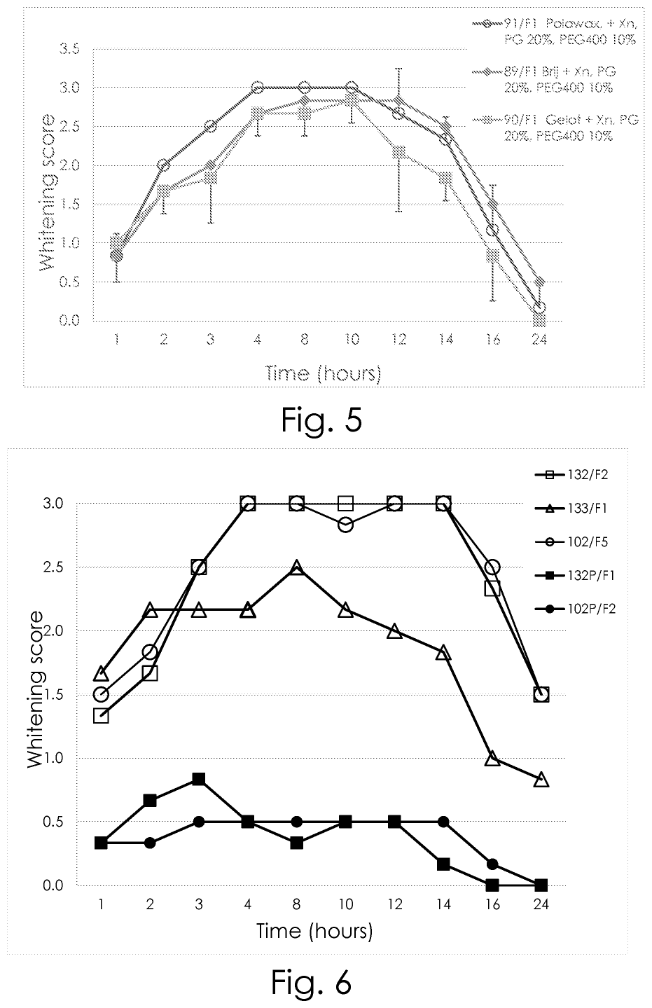

The skin whitening results for the W/O emulsions (Polawax, Brij and Gelot conceptions) are presented in FIG. 5 based on the average of 3 replicates.

The Polawax emulsion (19-0155.0091/F1) achieved the highest skin whitening with a score of 3 between 4 and 10 hours. A similar but slightly lower intensity profile was observed for the Gelot emulsion (19.0155-0090/F1).

The Brij-based composition showed some instability (despite the presence of 0.2% xanthan gum) and is considered unsatisfactory.

### Example 8: Evaluation of the Effect on Skin Whitening of Different Compositions Using an In Vivo Vasoconstriction Model

The compositions and physical stability data for the Gelot 64 and Polawax-based emulsions tested are described in Tables 17 and 18 below.

TABLE 17
Detailed compositions of Gelot 64 emulsions tested using the in vivo vasoconstriction model

| | Formula-Batch No. | | |
|---|---|---|---|
| Composition | 19.0155-0102/F4 | 19.0155-0102/F5 | 19.0155-0102P/F2 |
| Concept | GELOT | GELOT | GELOT |
| Batch size (g) | 200 | 200 | 200 |
| Justification of the formula | Formula 0090 + 1% BHA | Formula 0090 with 1% BHA for | Formula 0090P vehicle + 1% |
| | | stability in validated bottles. | BHA |
| Ingredient | % w/w | | |
| Brimonidine Tartrate | 1.5 | 1.5 | 0 |
| Purified water | 39.82 | 39.8 | 41.5 |
| Propylene Glycol | 20 | 20 | 20 |
| Glycerine 4810 Vegetale | 5 | 5 | 5 |
| PEG-400 SR | 10 | 10 | 10 |
| KOLLIDON VA 64 | 1 | 1 | 1 |
| XANTHAN GUM | 0.2 | 0.2 | 0.2 |
| METHYLPARABEN | 0.2 | 0.2 | 0.2 |
| PHENOXYETHANOL | 1 | 1 | 1 |
| GELOT 64 | 3 | 3 | 3 |
| KOLLIWAX SA | 3 | 3 | 3 |
| MIGLYOL 812N | 2.5 | 2.5 | 2.5 |
| ARLAMOL PS11E-LQ-(RB) | 5 | 5 | 5 |
| KOLLICREAM OA | 2.5 | 2.5 | 2.5 |
| KOLLIWAX CA | 4 | 4 | 4 |
| BHA | 1 | 1 | 1 |
| PROPYLPARABEN | 0.1 | 0.1 | 0.1 |
| DL Tocopherol | | | |
| NaOH Solution 10% | 0.18 | 0.2 | |
| Total | 100.00 | 100.00 | 100.00 |

Stability data

| | | | |
|---|---|---|---|
| Macroscopic aspect T0 | Thick, slightly yellow cream that | Yellowish cream that does | Thick white cream that does not |
| | does not run | not run | run |
| T1M 4° C./RT/40° C. | N/A | N/A | T1M Idem T0/Idem T0/Slight |
| | | | increase in viscosity |
| T3M 4° C./RT/40° C. | N/A | Idem T0/slightly more coloured | N/A |
| | | than at 4° C./slightly more | |
| | | coloured than at RT | |
| Microscopic aspect T0 | Big number of small | Very fine and homogeneous | Large number of small LCs |
| | LCs <10 μm. | base, many LCs | 5-10 μm. |
| T1M 4° C./RT/40° C. | N/A | N/A | T1M Idem T0/Idem T0 some |
| | | | bubbles/Idem T0 |
| T3M 4° C./RT/40° C. | N/A | Idem T0/idem T0, | N/A |
| | | LCs <10 μm/Idem T0, bubbled | |
| pH T0 | 4.81 | 4.96 | 5.01 |
| T1M 4° C./RT/40° C. | N/A | N/A | 4.77/4.70/4.74 |
| T3M 4° C./RT/40° C. | N/A | 4.70/4.70/4.70 | N/A |
| Viscosity (RV mobile 34-6 rpm- | | mob RV29 speed 30: 6633 | |
| 5 min) T0 | | cP/19.9% (on Mar. 7, 2020) | |
| T3M 4° C./RT/40° C. | | RT; 10,930 cP, 32.8% | |

LC—liquid crystals

TABLE 18
Detailed compositions of Polawax NF emulsions tested using the in vivo vasoconstriction model

| | Formula-Batch No. | | |
|---|---|---|---|
| Composition | 19.0155-0132/F2 | 19.0155-0132P/F1 | 19.0155-0133/F1 |
| Concept | POLAWAX | POLAWAX | POLAWAX |
| Batch size (g) | 200 | 200 | 200 |
| Justification of the formula | 19-0155.0091/F1 + 0.1% | 19-0155.0091P/F1 + 0.1% | 19-0155.0091/F1 + 1% |
| | Tocopherol | Tocopherol (vehicle) | Tocopherol |
| Ingredient | % w/w | | |
| Glycerine 4810 Vegetale | 4 | 4 | 4 |
| Propylene Glycol | 20 | 20 | 20 |
| PEG-400 SR | 10 | 10 | 10 |
| Purified water | 45.5 | 47.03 | 44.6 |
| Brimonidine Tartrate | 1.5 | | 1.5 |
| KOLLIDON VA 64 | 1 | 1 | 1 |
| XANTHAN GUM | 0.2 | 0.2 | 0.2 |
| POLAWAX NF | 10 | 10 | 10 |
| ARLAMOL PS11E-LQ-(RB) | 2.35 | 2.35 | 2.35 |
| DIMETHICONE (XIAMETER | 1 | 1 | 1 |
| PMX-200) | | | |
| KOLLICREAM OA | 2.5 | 2.5 | 2.5 |
| MARCOL 82 | 0.65 | 0.65 | 0.65 |
| BHA | | | |
| DL TOCOPHEROL | 0.1 | 0.1 | 1 |
| IP PARABENS | 1 | 1 | 1 |
| NaOH Solution 10% | 0.2 | | 0.2 |
| Citric acid solution 10% | | 0.17 | |
| Total | 100.00 | 100.00 | 100.00 |

Stability data

| | | | |
|---|---|---|---|
| Macroscopic aspect T0 | Slightly yellow. Smooth, shiny | White to off-white smooth and | Slightly yellow. Smooth, shiny |
| | cream that does not run | shiny cream that does not run | cream that does not run |
| T1M 4° C./RT/40° C. | T1M Idem T0/Surface | T1M Idem T0/white matte | T1M Idem T0/pearly |
| | thickening, pearlescent | aspect on surface/Idem T0 | appearance, fine |
| | effect/yellow colour on | | mottling/thicker and more |
| | surface, non-homogeneous | | coloured surface |
| T2M 4° C./RT/40° C. | N/A | T2M Idem T0/pearly mottling | T2M idem T0/idem T1M/Idem |
| | | aspect/thick cream, loss of | T1M |
| | | homogeneity | |
| T3M 4° C./RT/40° C. | N/A | T3M Idem T0/Idem | T3M idem T1M/Yellow ring |
| | | T2M/Idem T2M | on surface |
| Microscopic aspect T0 | Many little LCs | Very fine base, homogeneous. | Very fine base, many little |
| | | Globules <10 μm. Many little | LCs <10 μm |
| | | LCs. | |
| T1M 4° C./RT/40° C. | T1M idem T0/idem T0/idem | T1M Idem T0/fewer small | T1M Idem T1M at RT/less |
| | T0 | LCs, some large globules ~20 | clear LCs and some distorted, |
| | | μm/Idem T1M at RT | very thin base/bubbled form, |
| | | | large distorted LCs |
| T2M 4° C./RT/40° C. | N/A | T2M very thin base/few | T2M idem T1M/idem |
| | | bubbles, idem T1M/Bubbles, | T1M/very fine bubbled base, |
| | | heterogeneous base, less clear | small, less clear LCs |
| | | LCs | |
| T3M 4° C./RT/40° C. | N/A | T3M Idem T2M/Idem | T3M idem T1M/idem |
| | | T1M/Heterogeneous | T1M/Loss of homogeneity |
| pH T0 | 4.75 | 4.8 | 4.84 |
| T1M 4° C./RT/40° C. | 4.88/4.78/4.98 | 4.78/4.68/4.92 | 4.93/4.83/5.03 |
| T2M 4° C./RT/40° C. | N/A | 4.93/4.91/5.19 | 4.81/4.85/4.98 |
| T3M 4° C./RT/40° C. | N/A | 4.91/4.82/4.99 | 4.83/4.85/4.90 |

LC—liquid crystals

The Gelot-based compositions thus used are optimised versions of the Gelot 64 emulsion (19-0155-0090/F1 and 19.0155-0103/F1) while the Polawax emulsions are optimised variants of the Polawax emulsion (19-0155.0091/F1).

Each of the compositions was triple tested using the in vivo vasoconstriction protocol as described previously.

The results thus obtained are shown in FIG. 6. According to these results, formulations comprising 1.5% w/w brimonidine tartrate generate high levels of prolonged skin whitening indicative of a vasoconstriction.

The Gelot-based emulsions tested achieved maximum whitening performance with slightly lower performance than a Polawax 19-0155-0133/F1-based emulsion. This formula contains exactly the same concentration of an active agent as the other tested compositions and the other active Polawax formula (19-0155-0132/F2). The only difference is that composition 19-0155-0133/F1 contains 1.0% w/w tocopheral while 19-0155-0132/F2 contains 0.1% w/w tocopherol.

The Gelot 64 (19-0155-0102/F5) and Polawax (19-0155-0132/F2)-based emulsion formulations generated similar vasoconstriction profiles. The degree of vasoconstriction is similar, about 1-1.5 after application and at the maximum value after about 4 hours. The intensity of vasoconstriction remained around 3.0 until 14 hours after application and reduced to 1.0-1.5 after 24 hours.

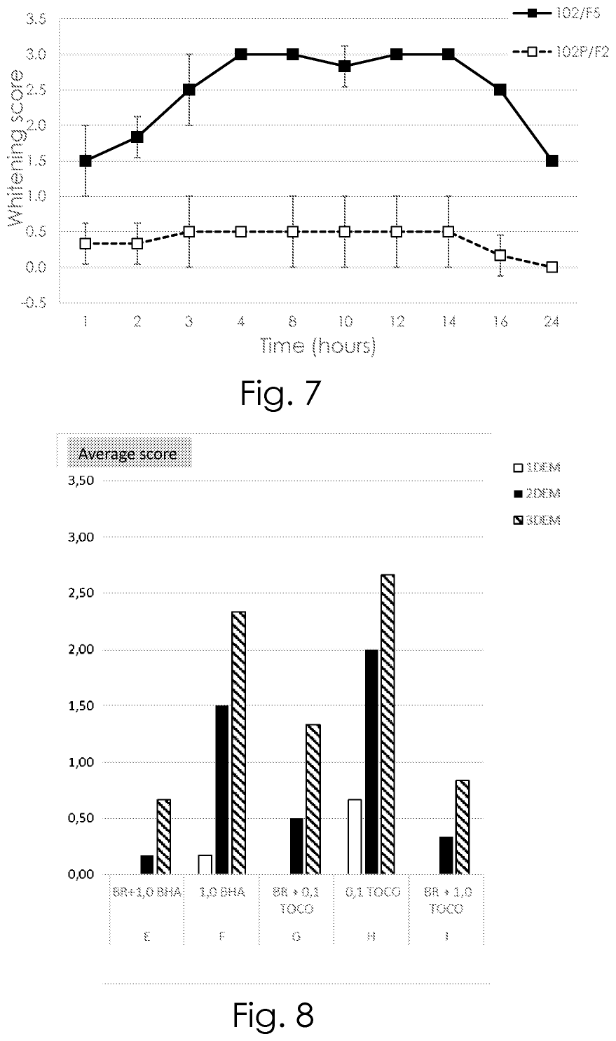

As an example, FIG. 7 shows the mean skin whitening profiles±the standard deviation of active formulation 19-0155-0102/F5 and corresponding vehicle 19-0155-0102P/F2 and also demonstrates the excellent reproducibility of the vasoconstriction test.

### Example 9: Evaluation of the Effectiveness on a UV-Induced Erythema Model

Active compositions comprising brimonidine tartrate and vehicles listed in Table 19 were evaluated using the UV-induced erythema model.

The compositions were applied using the protocol as specified below. This involved an application in the evening before UV irradiation and 2 hours before UV irradiation in three healthy volunteers.

Individual minimum erythemal doses (MED) for each subject were determined 24 hours before the experiment. Nine mini-zones were also marked on the dorsal trunk of each subject where the compositions were applied at a dose of 5 mg/cm2. UV doses were administered and equated to 1×MED, 2×MEDs (2MEDs) and 3×MEDs (3MEDs).

An experimental reading was taken 24 hours after irradiation using an investigator erythema score and using a Chromameter colorimeter.

TABLE 19

| Form of | | | | |
|---|---|---|---|---|
| dosage | Description | Main characteristics | Formula no. | Reference no. |
| Emulsion | Gelot 64 Liquid | Brimonidine tartrate 1.5% + | 19-0155.0102/F5 | E |
| | crystal emulsion | BHA 1.0% | (cf. Table 17) | |
| | | BHA 1.0% | 19-0155.0102P/F2 | F |
| | | | (cf. Table 17) | |
| | Polawax NF - | Brimonidine tartrate 1.5% + | 19-0155.0132/F2 | G |
| | Liquid crystal | Tocopherol 0.1% | (cf. Table 18) | |
| | emulsion | Tocopherol 0.1% | 19-0155.0132P/F1 | H |
| | | | (cf. Table 18) | |
| | | Brimonidine tartrate 1.5% + | 19-0155.0133/F1 | I |
| | | Tocopherol 1.0% | (cf. Table 18) | |

The average erythema scores for each composition are summarised in FIG. 8.

Active compositions containing 1.5% brimonidine tartrate demonstrated substantial reductions in erythema scores compared to vehicles. Additional benefits in terms of anti-erythematous effects were observed when 1.5% w/w brimonidine tartrate was combined with antioxidants. BHA and α-tocopherol were used as model antioxidants at concentrations of 0.1% and 1% w/w. Formulations containing 1% BHA or 1% α-tocopherol showed the most potent anti-erythematous effects and effectively inhibited 2 MEDs. Formula 19-0155.0102/F5 (liquid crystal emulsion 0/W Gelot 64) generated the strongest anti-erythema effect.

### Example 10: Performance of Reference Products on the Skin Whitening Model

The skin whitening (vasoconstriction) model was tested using reference pharmaceuticals known to induce skin whitening/vasoconstriction. The initial studies were carried out with:
MIRVASO® gel (0.5% w/w brimonidine tartrate)
Clobetasol propionate cream, 0.05% w/w
Norepinephrine hydrochloride solution (82 mg/ml in 70:30 ethanol:water) as used by Fahl (Effect of topical vasoconstrictor exposure upon tumoricidal radiotherapy. Int J Cancer; 135(4):981-988, 2014) and similar to the formulation used by Cleary et al. (Significant suppression of radiation dermatitis in breast cancer patients using a topically applied adregernic vasoconstrictor. Radiation Oncology, 2017).

The skin whitening model allows differentiation of the skin whitening capacity caused by several active agents applied in different formulations in terms of onset, intensity and duration of skin whitening.

This model has adequate reproducibility and a discriminatory performance for the formulation screening phase.

Clobetasol propionate cream was selected as a well-defined product/active agent involved in skin whitening. In fact, the effectiveness and in vivo bioequivalence of topical corticosteroids (1997 FDA guidance, https://www.fda.gov/media/70931/download) was assessed using this vasoconstriction effect.

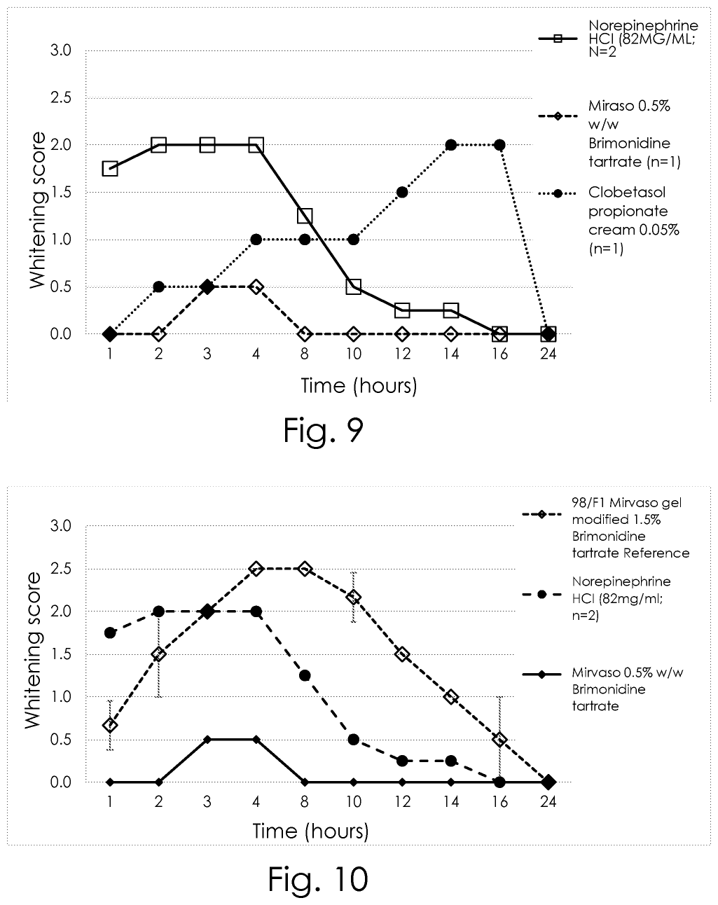

Skin whitening data for MIRVASO® gel, clobetasol propionate cream, 0.05% w/w and ephinephrine HCl solution (82 mg/ml in 70:30 Ethanol:water) are shown graphically in FIG. 9.

MIRVASO® gel did not general significant whitening. This was expected as it was designed for application to relatively thin and sensitive facial skin during the treatment of rosacea. As a general rule, these products do not contain high concentrations of potential skin penetrating agents due to the skin sensitivity of rosacea patients. In addition, the skin of the face is thinner than the skin of the chest and thus has a lower barrier to skin penetration and permeation. The intensity and duration of action are insufficient to meet the desired radiation-induced dermatitis requirements.

Norepinephrine (noradrenaline) solution produced rapid and intermediate skin whitening 1 hour after application; however, the effect began to fade after the 4-hour observation interval. Whitening was at 0.5 or less after 10 hours and returned to the observation starting point only after 16 hours. While the initial affects were promising, the duration and intensity of the effect are not sufficient.

In contrast to the polar norepinephrine molecule and its aqueous ethanol vehicle, clobetasol propionate cream exhibited a slow onset of action with a gradual increase from 2 hours to a peak whitening score of 2.0 between 14 and 16 hours. There is a rapid reduction in whitening from 16 hours and a return to the starting point at 24 hours. The slower onset of action of clobetasol whitening may be related to the physicochemical characteristics of the active agent, whereby its lipophilic nature results in reservoir formation and a more limited distribution in the viable epidermis compared to the more hydrophilic norepinephrine. It is also important to note that the pharmacodynamic mechanisms of vasoconstriction are also different for clobetasol propionate and norepinephrine.

Therefore, a modified MIRVASO formulation was prepared with a high dose of 1.5% w/w brimonidine tartrate (19-0155-0098/F1). This evaluation was conducted to assess the impact of an increased dose on skin whitening in a vehicle similar to MIRVASO.

The corresponding skin whitening results for the two formulae are shown in FIG. 10. Norepinephrine solution and commercially available MIRVASO® gel (0.5% brimonidine tartrate) are also included for comparison.

The modified MIRVASO gel (1.5% brimonidine tartrate 19-0155-0098/F1) caused a substantial increase in the intensity and duration of skin whitening compared to the commercially available MIRVASO® gel (0.5% brimonidine tartrate).

However, the intensity of skin whitening did not persist for a prolonged period of time as desired to solve the technical problem according to the invention.

This performance limitation is also coupled with the unsuitability of the MIRVASO vehicle in radiation dermatitis. As previously mentioned, the presence of titanium dioxide particles in the MIRVASO vehicle will interfere with and disrupt the radiation dose associated with the high energy electromagnetic waves used.

1. A composition in a form suitable for topical administration comprising:
a water base,
a vasoconstrictor chosen from brimonidine or salts thereof, wherein the vasoconstrictor is in a solvent-based phase comprising:
polyethylene glycol in combination with propylene glycol;
a hydrophilic film-forming agent chosen from a polyvinylpyrrolidone/vinyl acetate copolymer, polyvinylpyrrolidone in a non-crosslinked, crosslinked or acetate form, taken alone or in combination;
glycerine;
an emulsifier chosen from (i) the combination of PEG-75 stearate and glyceryl monostearate and (ii) the combination of polyoxyethylene-20 sorbitan monostearate and cetostearyl alcohol;
an oleic acid or an oleyl alcohol; and
an oil phase suitable for obtaining an emulsion comprising liquid crystals;
wherein the composition is an emulsion comprising liquid crystals.

2. The composition according to claim 1, wherein the hydrophilic film-forming agent is the polyvinylpyrrolidone/vinyl acetate copolymer.

3. The composition according to claim 1, wherein the oil phase comprises cetyl alcohol and stearyl alcohol, taken alone or in combination, and/or (i) a triglyceride ester of saturated caprylic and capric fatty acids saturated of coconut/palm kernel and of glycerol of vegetable origin, and (ii) a polypropylene glycol (PPG)-11 stearyl ether, taken alone or in combination.

4. The composition according to claim 3, wherein the oil phase comprises a combination of cetyl alcohol and stearyl alcohol.

5. The composition according to claim 3, wherein the oil phase comprises a combination of (i) the triglyceride ester of saturated caprylic and capric fatty acids of coconut/palm kernel and of glycerol of vegetable origin, and (ii) the PPG-11 stearyl ether.

6. The composition according to claim 1, further comprising xanthan gum.

7. The composition according to claim 6, wherein the xanthan gum is at a concentration of between 0.1% and 1.5% by weight of the total weight of the composition.

8. The composition according to claim 1, further comprising a natural or synthetic antioxidant, or a free radical scavenger.

9. The composition according to claim 8, wherein the antioxidant is chosen from the group consisting of butylated hydroxyanisole (BHA), DL-tocopherol, butylated hydroxytoluene (BHT), propaldehyde, ascorbate palmitate or glutathione, and combinations thereof.

10. The composition according to claim 1, wherein the brimonidine or salts thereof is at a concentration between 0.15% and 3.00% by weight of the total weight of the composition.

11. The composition according to claim 1, wherein the brimonidine or salts thereof is brimonidine tartrate.

12. The composition according to claim 1, further comprising a paraben chosen from the group consisting of methylparaben, propylparaben or isopropylparaben, phenoxyethanol, sodium benzoate, phenylethyl alcohol, ethylenediaminetetraacetic acid (EDTA), and combinations thereof.

13. (canceled)

14. (canceled)

15. The composition according to claim 1, wherein the brimonidine or salts thereof is present at a concentration between 0.50% and 2.50% by weight of the total weight of the composition.

16. The composition according to claim 1, wherein the brimonidine or salts thereof is present at a concentration between 0.75% and 1.50% by weight of the total weight of the composition.

17. The composition according to claim 1, wherein the brimonidine or salts thereof is present at a concentration between 1.00% and 1.50% by weight of the total weight of the composition.

18. The composition according to claim 9, wherein the antioxidant is a combination of butylated hydroxyanisole (BHA) and DL-tocopherol.

19. The composition according to claim 7, wherein the xanthum gum is at a concentration of between 0.2% and 1% by weight of the total weight of the composition.

20. A method of treating dermatitis caused by radiation from radiotherapy treatment, the method comprising:
providing a composition according to claim 1 to a radiotherapy treatment patient in need thereof, and
topically applying the composition to a radiotherapy treatment site of the patient.
